(19) United States
(12) Patent Application Publication
(10) Pub. No.: US 2009/0186918 A1
(43) Pub. Date: Jul. 23, 2009

(54) Triazole Compounds as Lipoxygenase Inhibitors

(76) Inventors: Benjamin Pelcman, Solna (SE); Andrei Sanin, Solna (SE); Peter Nilsson, Solna (SE); Hasse Kromann, Copenhagen (DK)

Correspondence Address: MORGAN LEWIS & BOCKIUS LLP, 1111 PENNSYLVANIA AVENUE NW, WASHINGTON, DC, 20004, US

(21) Appl. No.: 12/084,400

(22) PCT Filed: Oct. 27, 2006
(86) PCT No.: PCT/GB2006/004010

§ 371 Date: Mar. 27, 2009

Related U.S. Application Data
(60) Provisional application No. 60/731,481, filed on Oct. 31, 2005.

(51) Int. Cl. A61K 31/4709; C07D 401/12; A61K 31/4439

(52) U.S. Cl. 514/314; 546/169; 514/340; 546/268.4

(57) ABSTRACT

There is provided compounds of formula (I) wherein W is an optionally substituted aryl or heteroaryl group, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

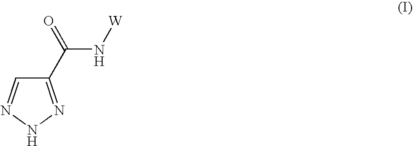

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomotology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

International patent application WO 00/034269 discloses various compounds including thiourea-containing 1,2,3-triazole-4-carboxylic acid amides. This document does not mention or suggest the use of such compounds in the treatment of inflammation.

Heteroaryl-based compounds including thiazoles have been disclosed in several publications. For example, international patent application WO 2005/007625 discloses anti-tuberculosis compounds that include triazoles; international patent application WO 2004/106324 discloses inter alia triazoles for use as herbicides; international patent applications WO 02/070483 and WO 03/016304 disclose various pest-controlling agents that include triazoles; US Patent No. 2002/009116 and international patent application WO 99/32454 disclose inter alia triazoles for use as Factor Xa inhibitors; international patent application WO 01/21160 discloses antiviral compounds that include triazoles. There is no disclosure in any of these documents of 1(N)-unsubstituted-1,2,3-triazole-4-carboxylic acid amides for use in treating inflammation and/or as inhibitors of lipoxygenases.

International patent applications WO 2004/080999, WO 2006/032851 and WO 2006/032852 all disclose various 3-amidopyrazoles for use in the treatment of inflammation. However, there is no disclosure or suggestion in any of these documents of 1,2,3-triazole-4-carboxylic acid amides.

International patent application WO 97/30034 discloses various 4-aminoquinazoline derivatives for use as antitumor agents. The document does not disclose or suggest compounds without such a substituent, nor does it mention or suggest the use of such compounds in the treatment of inflammation.

International patent application WO 2004/096795 discloses various heterocycles, including triazoles, as inhibitors of protein tyrosine kinaes, international patent application WO 02/092573 discloses various heterocycles for use as inhibitors of inter alia JNK3 protein kinases and international patent application WO 01/55115 discloses various aromatic amides that may be useful as activators of caspases and inducers of apoptosis. Accordingly, the compounds disclosed in these documents may be useful in the treatment of inter alia cancer. There is no disclosure or suggestion in any of these documents of the use of such compounds as inhibitors of lipoxygenases.

International patent application WO 97/19062 discloses various heterocycles for the treatment of skin related diseases and further mentions the use of such compounds in the treatment of various inflammatory diseases. However, there this document does not mention or suggest 3-amido triazoles.

JP Patent No. 10195063 discloses various 2-ethynylthiazole derivatives that may be employed as leukotriene antagonists, and may therefore be useful in the treatment of inflammation. However, this document does not mention or suggest compounds without such a substituent.

International patent application WO 2004/041789 discloses various compounds that may be useful as protein kinase inhibitors (and therefore useful in the treatment of inter alia autoimmune diseases). However, there is no specific disclosure of a 1,2,3)-triazole-4-carboxylic acid amide in this document.

International patent applications WO 03/068767, WO 03/037274, WO 96/18617, WO 2005/009954, WO 2005/009539, WO 2004/108133 and WO 2004/106305 all disclose various compounds, including triazoles, that may be useful in the treatment of inflammation. However, none of these documents specifically disclose 1(N)-unsubstituted 1,2,3-triazole-4-carboxylic acid amides.

### DISCLOSURE OF THE INVENTION

According to the invention there is provided a compound of formula I,

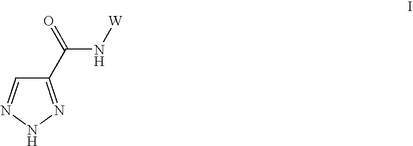

wherein
W represents an aryl or heteroaryl group, optionally substituted by one or more substituents selected from:

### 1) G¹;

2) aryl or heteroaryl, both of which are optionally substituted by one or more substituents selected from A¹, —N₃, —NO₂ and —S(O)ₚR^{6e}; and
3) heterocycloalkyl, which is optionally substituted by one or more substituents selected from A², —N₃, —NO₂ and ═O;
G¹ represents halo, R^{3a}, —CN, —C(O)R^{3b}, —C(O)OR^{3c}, —C(O)N(R^{4a})R^{5a}, —N(R^{4b})R^{5b}, N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, N(R^{3k})S(O)₂R^{3m}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r};
R^{3a} represents Clot allyl optionally substituted by one or more substituents selected from Z, F, Cl, —N(R^{6b})R^{6c}, —N₃, ═O and —OR^{6d};
R^{3b}, R^{3c}, R^{3h}, R³ⁿ and R^{4e} to R^{4h} independently represent H, Z or C₁₋₆ alkyl optionally substituted by one or more halo atoms or —OR^{6d};
R^{3d} to R^{3g}, R^{3k}, R^{3q}, R^{5a}, R^{5b}, R^{5d} and R^{5f} to R^{5h} independently represent H or C₁₋₆ alkyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h}, may be linked together to form a 3- to 6-membered ring, which ring optionally contains a further heteroatom (such as nitrogen or oxygen) in addition to the nitrogen atom to which these substituents are necessarily attached, and which ring is optionally substituted by ═O or C₁₋₆ alkyl optionally substituted by one or more fluoro atoms;
R³ⁱ, R^{3j}, R^{3m}, R^{3p} and R^{3r} independently represent Z or C₁₋₆ alkyl optionally substituted by one or more substituents selected from B¹;
R⁴ⁱ and R⁵ⁱ independently represent H or C₁₋₆ alkyl optionally substituted by one or more substituents selected from B²;
Z represents, on each occasion when mentioned herein:
a) heterocycloalkyl optionally substituted by one or more substituents selected from A³ and ═O;
b) aryl or heteroaryl both of which are optionally substituted by one or more substituents selected from A⁴, —N₃, —NO₂ and —S(O)_{q}R^{7e};
A¹, A², A³ and A⁴ independently represent halo, —R^{6a}, —CN, —N(R^{6b})R^{6c} or —OR^{6d};
R^{6b} to R^{6d} independently represent, on each occasion when mentioned herein, H or C₁₋₆ alkyl optionally substituted by one or more substituents selected from B3;
R^{6a}, R^{6e} and R^{7e} independently represent C₁₋₆ alkyl optionally substituted by one or more substituents selected from B⁴; or
R^{6b} and R^{6c} may be linked together to form a 3- to 6-membered ring, which ring optionally contains a further heteroatom (such as nitrogen or oxygen) in addition to the nitrogen atom to which these substituents are necessarily attached, and which ring is optionally substituted by ═O or C₁₋₆ alkyl optionally substituted by one or more fluoro atoms;
B¹, B², B³ and B⁴ independently represent F, Cl, —OCH₃, —OCH₂CH₃, —OCHF₂, —OCH₂CF₃, —OCF₃ or —OCF₂CF₃; and
m, p and q independently represent 0, 1 or 2,
or a pharmaceutically-acceptable salt thereof,
provided that:
(A) when W represents a phenyl group substituted by one G¹ substituent at the ortho position, G¹ represents R^{3a}, R^{3a} represents ethynyl substituted by Z, Z represents 2-thiazolyl substituted in the 4-position by A⁴ and A⁴ represents R^{6a}, then R^{6a} does not represent cyclobutyl;
(B) when W represents a 6-quinazolinyl group substituted in the 4-position by G¹,

G¹ represents —N(R^{4b})R^{5b}, R^{5b} represents H and R^{4b} represents Z, then Z does not represent 3-chloro-4-fluorophenyl,

which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alynyl group).

The term “halo”, when used herein, includes fluoro, chloro, bromo and iodo.

Heterocycloalkyl groups that may be mentioned include monocyclic or bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include 7-azabicyclo[2.2.1]heptanyl, 6-azabicyclo[3.1.1]hept-anyl, 6-azabicyclo[3.2.1]octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl oxetanyl oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl, thietanyl, thiiranyl, thiolanyl thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the other substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

Aryl groups that may be mentioned include C₆₋₁₄ (e.g. C₆₋₁₀) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an atom of the aromatic ring.

Heteroaryl groups that may be mentioned include those which have between 5 and 14 (e.g. between 5 and 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzothiadiazolyl (including 2,3,1-benzothiadiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are bicyclic or tricyclic, they are linked to the rest of the molecule via an atom of the aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which W is substituted by two or more substituents, those substituents may be the same or different. For example, when W is substituted by two substituents, and the substituents are both —C(O)R^{3b} in which R^{3b} is a C₁₋₆ alkyl group, the respective alkyl groups may be the same or different. Similarly, when W is substituted by more than one substituent as defined herein, the identities of those individual substituents are not to be regarded as being interdependent. For example, when one substituent represents —C(O)R^{3b} and the other substituent represents —C(O)R^{3c}, and R^{3b} and R^{3c} both represent C₁₋₆ alkyl substituted by —OR^{6d}, the identities of the two —OR^{6d} groups are not to be regarded as being interdependent.

Compounds of the invention that may be mentioned include those in which:

W is not substituted by phenyl, 4H-[1,2,4]triazol-4-yl, pyridyl or indolizinyl;
W does not represent a pyrimidinyl (e.g. 5-pyrimidinyl) group;
W does not represent a pyrazolyl group;
W does not represent a pyridyl (e.g. a 2-pyridyl) group;
W does not represent a 6,5-bicyclic group in which the 6-membered ring is aromatic and the 5-membered ring is non-aromatic;

when W represents a 2-quinolinyl or 1-isoquinolinyl group, both of which are substituted (e.g. at the S-position) by a —C(O)N(R^{4a})R^{5a} and/or a —N(R^{3d})C(O)R^{4c} group, and R^{3d} and R^{4a} each represent hydrogen, then R^{5a} and/or R^{4c} (as appropriate) do/does not represent a C₃₋₆ alkyl (e.g. a C₃₋₆ cycloalkyl or C₄₋₆ part cyclic alkyl) group;

when W represents 2-pyridyl or 2-pyrimidinyl, both of which are substituted (e.g. in the 4-position) by a heteroaryl group, then such a heteroaryl group does not represent optionally substituted 4-pyrazolyl.

Further compounds of the invention that may be mentioned include those in which:

when W (for example when W is phenyl) is substituted at the ortho position (relative to the point of attachment of W to the —N(H)C(O)— group of the compound of formula I), then the substituent is selected from:

### 1) G¹;

2) aryl or heteroaryl, both of which are optionally substituted by one or more substituents selected from A¹, —N₃, —NO₂ and —S(O)ₚR^{6e}—; and
3) heterocycloalkyl, which is optionally substituted by one or more substituents selected from A², —N₃, —NO₂ and ═O,
in which the heteroaryl or heterocycloalkyl group does not contain a nitrogen atom and G¹ represents halo, —R^{3a}, —CN, C(O)R^{3b}, —C(O)OR^{3c}, —C(O)N(R^{4a})R^{5a}, —N₃, —NO₂, —OR^{3h}, —OC(O)N(R^{4g})R^{5g}, —OS(O)₂R³ⁱ, S(O)ₘR^{3j}, —OC(O)R³ⁿ, —OC(O)OR^{3p}, —S(O)₂N(R^{4h})R^{5h}, —S(O)₂OH, —P(O)(OR^{4j})(OR⁵ⁱ) or —C(O)N(R^{3q})S(O)₂R^{3r}.

Yet further compounds of the invention that may be mentioned include those in which:

when W (for example when W is phenyl) is substituted at the ortho position (relative to the point of attachment of W to the —N(H)C(O)— group of the compound of formula I), then the substituent is selected from:

### 1) G¹;

2) aryl or heteroaryl, both of which are substituted by one or more substituents selected from A¹, —N₃, —NO₂ and —S(O)ₚR^{6e}; and
3) heterocycloalkyl, which is substituted by one or more substituents selected from A², —N₃, —NO₂ and ═O,
in which A¹ and A² independently represent —R^{6a}, —CN, —N(R^{6b})R^{6c} or —OR^{6d} and G¹ represents halo, —CN, —C(O)R^{3b}, —C(O)OR^{3c}, —C(O)N(R^{4a})R^{5a}, —N(R^{4b})R^{5b}, —(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —C(O)N(R^{4g})R^{5g}, —OS(O)₂R³ⁱ, —N(R^{3k})S(O)₂R^{3m}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r}.

Yet further compounds of the invention that may be mentioned include those in which R^{4b} and R^{5b} are not linked together as defined herein.

Further compounds of the invention that may be mentioned include those in which:

R^{6a} represents acyclic C₁₋₆ alkyl optionally substituted by one or more substituents selected from B⁴;

R^{6a} represents C₁₋₃ alkyl or C₅₋₆ alkyl, both of which are optionally substituted by one or more substituents selected from B⁴;

A⁴ represents halo, —CN, —N(R^{6b})R^{6c} or —OR^{6d};
when Z represents heteroaryl, then it does not represent thiazolyl (e.g. 2-thiazolyl);
when Z represents heteroaryl (such as thiazolyl (e.g. 2-thiazolyl)), then such a group is substituted by one or more substituents selected from A⁴, —N₃, —NO₂ and —S(O)_{q}R^{7c}, in which A⁴ represents halo, —CN, —N(R^{6b})R^{6c} or —OR^{6d};
R^{3a} represents C₁₋₆ alkyl optionally substituted by one or more substituents selected from F, Cl, —N(R^{6b})R^{6c}, —N₃, ═O and OR^{6d};
when W represents heteroaryl, then it does not represent quinolinyl (e.g. 6-quinazolinyl);
when W represents 6-quinazolinyl, then such a group is not substituted in the 4-position (e.g. by G¹, for example when G¹ represents —N(R^{4b})R^{5b}); R^{4b} represents H or C₁₋₆ alkyl optionally substituted by one or more halo atoms or —OR^{6d};
G¹ represents halo, —R^{3a}, —CN, —C(O)R^{3b}, —C(O)OR^{3c}, —C(O)N(R^{4a})R^{5a}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)N(R^{4f})R^{5f}, —OR^{3h}, —OC(O)N(R^{4g})R^{5g}, —OS(O)₂R³ⁱ, —S(O)R^{3j}, —N(R^{3k})S(O)₂R^{3m}, —OC(O)R³ⁿ, —OC(O)OR^{3p}, —S(O)₂N(R^{4b})R^{5h}, —S(O)₂OH, —P(O)(OR⁴ⁱ)(OR⁵ⁱ) or —C(O)N(R^{3q})S(O)₂R^{3r}.

Preferred compounds of the invention include those in which W represents an optionally substituted phenyl, naphthyl, pyrrolyl, furanyl, thienyl, pyrazolyl, imidazolyl, oxazolyl, isoxazolyl, thiazolyl, pyridyl (e.g. 2-pyridyl, 3-pyridyl or 4-pyridyl), indazolyl indolyl, indolinyl, isoindolinyl, oxindolyl, quinolinyl, 1,2,3,4-tetrahydroquinolinyl, isoquinolinyl 1,2,3,4-tetrahydroisoquinolinyl, quinolizinyl, benzofuranyl, isobenzofuranyl, chromanyl, benzothienyl pyridazinyl, pyrimidinyl, pyrazinyl, indazolyl, benzimidazolyl, quinazolinyl, quinoxalinyl (e.g. 2-quinoxalinyl), 1,3-benzodioxolyl, benzothiazolyl, 1,4-benzodioxanyl, 1,3,4-oxadiazolyl or 1,3,4-thiadiazolyl, group.

Particularly preferred values of W include optionally substituted thiazolyl (e.g. 2-thiazolyl), 1,3-benzodioxolyl, pyrimidinyl (e.g. 2-pyrimidinyl) or, more preferably, optionally substituted quinoxalinyl (e.g. 2-quinoxalinyl), preferably, quinolinyl (e.g. 4-quinolinyl or, more preferably, 3-quinolinyl) and, more preferably, phenyl or pyridyl (e.g. 3-pyridyl or, more preferably, 2-pyridyl).

Preferred compounds of the invention include those in which:

R^{3k} and R^{3q} independently represent H;
R^{3m} and R^{3r} independently represent Z, in which Z represents aryl (e.g. phenyl), heteroaryl (e.g. pyridyl), which latter two groups are optionally substituted as defined herein, or C₁₋₆ (e.g. C₁₋₃) alkyl (e.g. methyl) optionally substituted by one or more fluoro atoms (so forming, for example, a trifluoromethyl group);
R^{3p} and R³ⁿ (when R³ⁿ represents optionally substituted allyl) independently represent C₁₋₃ (e.g. C₁₋₂) alkyl optionally substituted by one or more fluoro atoms; when Z represents an aryl or heteroaryl group, both of these are optionally substituted by one or more substituents selected from A⁴;
A¹, A², A³ and A⁴ independently represent halo (e.g. chloro or, particularly, fluoro), —R^{6a} or —OR^{6d};
when any of R^{6a}, to R^{6e}, or R^{7e} represent optionally substituted C₁₋₆ alkyl, then that alkyl group is an optionally substituted C₁₋₄ (e.g. C₁₋₂) alkyl group;
when R^{6b} and R^{6c} are linked together, they form a 5- to 6-membered ring, which ring optionally contains a flirter heteroatom (such as nitrogen or oxygen) and is optionally substituted by methyl, —CHF₂, —CF₃ or ═O (so forming, for example, a pyrrolidinyl, piperidinyl, morpholinyl or a piperazinyl (e.g. 4-methylpiperazinyl) ring);
B¹, B², B³ and B⁴ independently represent F or Cl;
m, p and q independently represent 2.

More preferred compounds of the invention include those in which:

W is optionally substituted by between 1 and 4 substituents (e.g. aryl or G¹);
G¹ represents N₃ or, more preferably, halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —NO₂, —N(R^{3g})S(O)₂N(R^{4f})R^{5f}, —OR^{3h}, —OC(O)N(R^{4g})R^{5g}, —OS(O)R³ⁱ, —S(O)ₘR^{3j} or —S(O)₂N(R^{4h})R^{5h};
when any of the pairs R^{4a} and R^{5a}, R^{4b} and R^{5b}, R^{4d} and R^{5d}, R^{4f} and R^{5f}, R^{4g} and R^{5g}, or R^{4h} and R^{5h}, are linked together, they form a 5- to 6-membered ring, which ring optionally contains a further heteroatom (such as nitrogen or oxygen) and is optionally substituted by methyl, —CHF₂, —CF₃ or ═O (so forming, for example, a pyrrolidinyl, piperidinyl, morpholinyl or a piperazinyl (e.g. 4-methylpiperazinyl) ring).

Further preferred compounds of the invention include those in which:

R^{3a} represents C₁₋₆ alkyl optionally substituted by one or more substituents selected from F and —OR^{6d};
R^{3b}, R^{3c}, R^{3h}, R^{4a} to R^{4h}, R^{5a}, R^{5b}, R^{5d}, R^{5f} to R^{5h} independently represent H or optionally substituted C₁₋₄ alkyl (e.g. methyl) or the relevant pairs (i.e. R^{4a} and R^{5a}, R^{4b} and R^{5b}, R^{4d} and R^{5d}, R^{4f} and R^{5d}, R^{4f} and R^{5f}, R^{4g} and R^{5g} and R^{4h} and R^{5h}) may be linked together as hereinbefore defined;
R^{3d} to R^{3g} independently represent C₁₋₄ (e.g. C₁₋₂) alkyl (such as a methyl) or, more particularly, H;
R³ⁱ and R^{3j} independently represent C₁₋₄ alkyl optionally substituted by one or more B¹ substituents;
B¹ represents F (thus R³ⁱ and R^{3j} may represent a CH₃ or CF₃ group); when any one of R^{3b}, R^{3c} to R^{3b}, R^{4a} to R^{4h}, R^{5a}, R^{5b}, R_{5d}, R^{5f} to R^{5h} represents alkyl, preferred optional substituents include —OCH₃ and, especially, F.

Yet more preferred compounds of the invention include those in which:

when W is substituted, then it is substituted by one to three substituents selected from G¹;
R^{3a} represents C₁₋₃ (e.g. C₁₋₂) alkyl (e.g. isopropyl or, more particularly, methyl or ethyl) optionally substituted by one or more fluoro atoms;
R^{3h} represents hydrogen or C₁₋₄ (e.g. C₁₋₂) alkyl (e.g. methyl or ethyl) optionally substituted by one or more fluoro atoms (so forming, for example, a —CF₃ group);
R^{4b} and R^{5b} independently represent C₁₋₂ alkyl (e.g. methyl or ethyl);
G¹ represents F, Cl, —CH₃, —CH₂CH₃, —CHF₂, —CF₃, —CH₂CF₃, —CN, —N(CH₃)₂, —N(CH₂CH₃)₂, —NO₂, —OH, —OCH₃, —OCH₂CH₃, —OCH₂CF₃, —OCHF₂, —OCF₃ and —OCF₂CF₃.

Preferred optional substituents on W include:

optionally substituted aryl (e.g. phenyl);
—N(R^{3f})C(O)OR^{4e}; preferably,
—S(O)₂N(R^{4h})R^{5h}; or, more preferably,
halo (e.g. bromo or, preferably, fluoro or chloro);

### —R^{3a};

### —OR^{3h};

### —NO₂;

R^{3a} represents n-propyl, ethyl or, more preferably, isopropyl or, preferably, methyl, which groups are optionally substituted by one or more fluoro atoms (so forming, for example, a —CF₃ group);
R^{3f} represents H;
R^{3h} represents trifluoromethyl, ethyl, propyl (e.g. n-propyl), butyl (e.g. n-butyl) or, more preferably, methyl;
R^{4e} represents C₁₋₄ alkyl (e.g. t-butyl), which group may be substituted by one or more halo atoms but is preferably unsubstituted;
R^{4h} and R^{5h} independently represent H, methyl or ethyl.

Thus, preferred optional substituents on W include phenyl, bromo, ethyl, propyl, —NHC(O)Ot-butyl, ethoxy, propoxy (e.g. n-propoxy), butoxy (e.g. n-butoxyl), trifluoromethoxy, particularly —S(O)₂NH₂, —S(O)₂N(CH₃)H, —S(ON(CH₃)₂, —S(O)₂N(CH₂CH₃)₂, isopropyl and, more particularly, fluoro, chloro, methyl, methoxy, —NO₂ and trifluoromethyl.

Preferred compounds of the invention include those in which:

W is a 5-membered monocyclic or 9-membered bicyclic ring or, more preferably, a 6-membered monocyclic ring or a 10-membered bicyclic ring;
when W is a noncyclic 5-membered ring, it is a heteroaryl ring containing at least one heteroatom (e.g. nitrogen) and a further optional heteroatom (e.g. sulfur), so forming, for example a thiazolyl (e.g. thiazol-2-yl) group;
when W is a monocyclic 6-membered ring, it is a phenyl group or a heteroaryl group preferably containing one or two (e.g. one) heteroatom (e.g. nitrogen) so forming, for example, a pyridyl group;
when W is phenyl, it is substituted by at least one substituent (e.g. in the 3- or, more preferably, the 2- or 4-position) or, preferably, at least two (e.g. two or three) substituents. When substituted by two substituents, preferred positions include the 2- and 3-, 3- and 5-, 2- and 6- or, more preferably, 2- and 5-, 3- and 4- or, more particularly, the 2- and 4-positions. When substituted by three substituents, and the first two substituents are in the 2- and 4-position, the third substituent is preferably in the 6- or, more preferably, 3- or 5-position. Preferred substituents in the 2-position of such phenyl rings include —S(O)₂NH₂, —S(O)₂N(CH₃)H, —S(O)₂N(CH₃)₂, isopropyl, preferably, trifluoromethyl, methoxy, —NO₂ and, more preferably, fluoro, chloro and methyl. Preferred substituents in the 4-position of such phenyl rings include methyl, trifluoromethoxy or, more preferably, —S(O)₂NH₂, —S(O)N(CH₃)H, —S(O)₂N(CH₃)₂, —S(O)₂N(CH₂CH₃)₂, preferably, —NO₂ and, more preferably, halo (e.g. bromo or, more preferably, fluoro and chloro) and trifluoromethyl. Other preferred substituents in the 3-, 5- and 6-positions include fluoro, chloro, bromo, methyl, ethyl, isopropyl, trifluoromethyl and methoxy;
when W is a monocyclic heteroaryl ring, it is substituted in the ortho-, meta- or, more preferably, para-position relative to the point of attachment of the monocyclic heteroaryl ring to the 3-amido group of the compound of formula I (provided that the para-position is not a heteroatom);
when W is a 9-membered bicyclic ring, it is a group in which the first ring (attached to the triazole-3-amido group) is aromatic, for example a 6-membered ring such as phenyl, and the second ring is non-aromatic, for example a 5-membered ring, e.g. containing one or two heteroatoms (e.g. oxygen heteroatoms), so forming, for example a dioxolyl (e.g. a [1,3]dioxolyl) group. Such groups may be substituted but are preferably unsubstituted;
when W is a 10-membered bicyclic ring, it is a bicyclic heteroaryl group in which both rings are aromatic and which group preferably contains one or two heteroatoms (e.g. nitrogen). Such heteroatoms are preferably in the first ring of the bicycle (i.e. that which is attached to the amido group of the compound of formula I). Such groups are preferably attached via the 2-, 3- or 4-position of the heteroaryl group and are unsubstituted or, more preferably, substituted by one or more substituent (e.g. one) selected from trifluoromethyl and, preferably, halo (e.g. fluoro or chloro), attached to, for example, the 6-, 7- or 8-position (provided that the substituent is not attached to a heteroatom of an aromatic ring).

For the avoidance of doubt, when phenyl rings are substituted, the relative position of the substituents refers to the relative position of the substituent in relation to the point of attachment of the phenyl ring. For example, the 2-, 3- and 4-positions refer to the ortho-, meta- and para-substituents, respectively (and the 5- and 6-positions refer to the alternative meta- and ortho-substituents, respectively).

When W is substituted by optionally substituted heterocycloalkyl, aryl or heteroaryl, then preferred values of such heterocycloalkyl, aryl or heteroaryl groups include optionally substituted 1-pyrrolidinyl, 1-piperidinyl, 4-morpholinyl, 1-piperazinyl, indolyl (e.g. 4-indolyl), oxadiazolyl, oxazolyl, phenyl, quinolinyl (e.g. 3-quinolinyl), pyrazolyl (e.g. 3-pyrazolyl), pyridyl (e.g. 2-pyridyl), tetrazolyl, thiadiazolyl, thiazolyl, thienyl and triazolyl (e.g. 1,2,4-triazol-3-yl). Preferred substituents on such groups include fluoro, chloro, methyl, trifluoromethyl, methoxy, trifluoromethoxy and/or, when such a group is heterocycloalkyl, ═O.

Particularly preferred values of Z include optionally substituted indolyl (e.g. 4-indolyl), oxadiazolyl, oxazolyl, quinolinyl (e.g. 3-quinolinyl), pyrazolyl (e.g. 3-pyrazolyl), thiadiazolyl, thiazolyl, thienyl and, more particularly, phenyl and pyridyl (e.g. 2-pyridyl). Preferred substituents on such Z groups include fluoro, chloro, methyl, trifluoromethyl, methoxy, trifluoromethoxy and/or, when Z represents a heterocycloalkyl group, ═O.

Preferred compounds of the invention also include those in which:

when W represents a quinolinyl group, it is unsubstituted or substituted by one halo (e.g. fluoro or chloro) substituent, for example at the 6, 7 or 8-position;

when W represents a pyridyl group, it may be substituted by two substituents, or is preferably substituted by one substituent, for example at the para position relative to the point of attachment of the pyridyl group (to the triazole-3-amido group), selected from bromo, nitro, methyl, ethyl, propyl, methoxy, ethoxy, propoxy (e.g. n-propoxy), butoxy (e.g. n-butoxy), phenyl, —N(H)C(O)Ot-butyl or, more preferably, chloro, fluoro and trifluoromethyl;

when W represents phenyl, it is unsubstituted or, more preferably, substituted as hereinbefore defined by 1 to 3 substituents;
when W represents a thiazolyl (e.g. thiazol-2-yl) group, it is preferably substituted, for example at the 5-position, by at least one (e.g. one) chloro group;
when W represents a pyrimidinyl (e.g. pyrimid-2-yl) group, it is unsubstituted or substituted, for example at the 4-position, by at least one (e.g. one) methyl group;
when W represents benzodioxolyl (e.g. benzo[1,3]dioxol-5-yl), it is preferably unsubstituted.

More preferred compounds of the invention that may be mentioned include those in which:

when W represents a substituted pyrid-2-yl group, it is preferably substituted by at least one (e.g. one or two) substituent, selected from bromo, nitro, methyl, ethyl, propyl, methoxy, ethoxy, propoxy (e.g. n-propoxy), butoxy (e.g. n-butoxy), —N(H)C(O)Ot-butyl, chloro, fluoro and trifluoromethyl;

when W represents a substituted pyrid-3-yl group, it is preferably substituted by at least one substituent (e.g. one or two) selected from methyl, methoxy, phenyl. Preferred substitution positions on 3-pyridyl groups include the 2-, 5- and 6-positions.

Preferred compounds of the invention include those in which W represents 2-chloro-4,6-difluorophenyl, 4-fluoro-3-methylphenyl, 2,3,4-trifluorophenyl, 2,3-dichlorophenyl, 2-chloro-5-methylphenyl, 3,5-dichlorophenyl, 2,4-bis(trifluoromethyl)phenyl, 2-fluoro-5-methylphenyl, 2-chloro-6-trifluoromethylphenyl, 5-chloro-2-methylphenyl, 2-methylsulfamoylphenyl, 2-dimethylsulfamoylphenyl, 2,4,6-trifluorophenyl, 3,5-difluorophenyl, 3,4-difluorophenyl, 2-fluoro-3-trifluoromethylphenyl, 2,5-difluorophenyl, 2,6-dichloro-4-fluorophenyl; 2-fluoro-5-trifluoromethylphenyl, 3-fluoro-4-methylphenyl, 3-chloro-4-methylphenyl, 3-fluoro-5-trifluoromethylphenyl, 4-chloro-2-methylphenyl, 3-trifluoromethyl-4-methylphenyl, 3,4-dichlorophenyl, 4-trifluoromethoxyphenyl, 5-fluoro-2-methylphenyl, 4-chloro-3-trifluoromethylphenyl, 2,6-dichloro-4-trifluoromethylphenyl, 3-chloro-4-fluorophenyl, 3-trifluoromethylphenyl, 3-chloro-2-methylphenyl, 4-fluoro-3-trifluoromethylphenyl, 2,6-diisopropylphenyl, 3,5-bis(trifluoromethyl)phenyl, 2-fluoro-6-trifluoromethylphenyl, 5-bromopyrid-2-yl, 5-nitropyrid-2-yl, 6-methoxypyrid-2-yl, 6-bromopyrid-2-yl, 4-trifluoromethylpyrid-2-yl, 4-methylpyrid-2-yl, 5-methylpyrid-2-yl, 5-ethyl-6-methylpyrid-2-yl, 3-chloro-5-trifluoromethylpyrid-2-yl, 5,6-dimethylpyrid-2-yl, 5-methoxypyrid-2-yl, 5,6-dimethoxypyrid-2-yl, 6-methylpyrid-2-yl, 4,6-dimethylpyrid-2-yl, 3,5-dichloropyrid-2-yl, 3-methoxypyrid-2-yl, 5-butoxypyrid-2-yl, 5-ethoxypyrid-2-yl, 5-propoxypyrid-2-yl, 5-propylpyrid-2-yl, 5-ethylpyrid-2-yl, 6-trifluoromethylpyrid-2-yl, 5-(NH—C(O)Ot-butyl)pyrid-2-yl, 2,5-dichloropyrid-3-yl, 5-methylpyrid-3-yl, 6-methoxy-5-methylpyrid-3-yl, 5-phenylpyrid-3-yl, 5-chlorothiazol-2-yl, benzo[1,3]dioxol-5-yl, pyrimidin-2-yl or 4-methylpyrimidin-2-yl. However, more preferred compounds of the invention include those in which W represents quinolin-4-yl, unsubstituted phenyl, 4-isopropylphenyl, 4-diethylsulfamoylphenyl, quinoxalin-2-yl, 4-sulfamoylphenyl, 4-methylsulfamoylphenyl, 4-dimethylsulfamoylphenyl, 2,4-dichloro-6-methylphenyl, 8-fluoroquinolin-3-yl, 8-chloroquinolin-3-yl, 2-fluoro-6-trifluoromethylphenyl, preferably, quinolin-3-yl, 6-fluoroquinolin-3-yl, 7-fluoroquiolin-3-yl, 2,4-dimethoxyphenyl, 4-chloro-2,5-dimethoxyphenyl, 2,4,6-trichlorophenyl, 2-trifluoromethylphenyl, 4-nitrophenyl or, more preferably, 2-chloro-4-fluorophenyl, 2,4-dichlorophenyl, 4-fluorophenyl, 2,3,4-trichlorophenyl, 3,4-dichlorophenyl, 2-chlorophenyl, 2,4,5-trichlorophenyl, 2,4-dimethylphenyl, 2,5-dichlorophenyl, 4-chloro-3-methylphenyl, 4-chloro-2-methoxyphenyl, 2,4-dichloro-3-methylphenyl, 2-nitro-4-trifluoromethylphenyl, 4-fluoro-2-trifluoromethylphenyl, 4-chloro-2-trifluoromethylphenyl, 4-chloro-2-fluorophenyl, 2-chloro-4-trifluoromethylphenyl, 5-chloropyrid-2-yl, 5-fluoropyrid-2-yl or 5-trifluoromethylpyrid-2-yl.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) Reaction of 1,2,3-triazole-4-carboxylic acid, or a N-protected and/or O-protected (e.g. ester) derivative thereof, with a compound of formula II,

WNH₂ II

wherein W is as hereinbefore defined under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethyl amine, 1,8-diaza-bicyclo[5.4.0]undec-7-ene, sodium hydroxide, N-ethyldiisopropylamine, N-(methylpolystyrene)-4-(methylamino)pyridine, butyllithium (e.g. n-, s- or t-butyl-lithium) or mixtures thereof), an appropriate solvent (e.g. tetrahydrofuran, pyridine, toluene, dichloromethane, chloroform, acetonitrile, dimethylformamide, dimethylsulfoxide, water or triethylamine) and a suitable coupling agent (e.g. 1,1′-carbonyldiimidazole, N,N′-dicyclohexylcarbodiimide, 1-(3-dimethylaminopropyl)-3-ethylcarbodimide (or hydrochloride thereof), N,N-disuccinimidyl carbonate, benzotriazol-1-yloxytris(dimethylamino)-phosphonium hexafluorophosphate, 2-(1H-benzotriazol-1-yl)-1,1,3,3-tetramethyluronium hexafluorophosphate, benzotriazol-1-yloxytris-pyrrolidinophosphonium hexafluorophosphate, bromo-tris-pyrrolidinophosphonium hexafluorophosphate, 2-(1H-benzotriazol-1-yl)-1,1,3,3-tetramethyluronium tetrafluorocarbonate, 1-cyclohexylcarbodiimide-3-propyloxymethyl polystyrene, O-(7-azabenzotriazol-1-yl)-N,N,N′,N′-tetamethyluronium hexafluorophosphate or O-benzotriazol-1-yl-N,N,N′,N′-tetramethyluronium tetrafluoroborate). Alternatively, 1,2,3-triazole-4-carboxylic acid may first be activated by treatment with a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. This activated intermediate may then be reacted with a compound of formula II under standard conditions, such as those described above. The skilled person will appreciate that when compounds of formula II are liquid in nature, they may serve as both solvent and reactant in this reaction. Alternative methods of performing this step include reaction of an O-protected derivative (e.g. an ethyl ester) of 1,2,3-triazole-4-carboxylic acid with a compound of formula II, which latter compound may first be treated with an appropriate reagent (e.g. triethylaluminium), for example in an inert atmosphere and in the presence of a suitable solvent (e.g. dichloromethane).
(ii) Reaction of 1,2,3-triazole-4-carboxylic acid amide, or a N-protected (e.g. at the triazole nitrogen) derivative thereof with a compound of formula III,

W-L¹ II

wherein L¹ represents a suitable leaving group, such as halo (e.g. chloro, bromo and iodo), —OSO₂CF₃, —B(OH)₂, —Sn(R^{z})₃ (wherein R^{z} is C₁₋₆ alkyl and preferably, methyl or butyl), —Pb(OC(O)CH₃)₃, —Bi(W)₂, —Bi(W)₂(OC(O)CH₃)₂, —Bi(W)₂(OC(O)CF₃)₂ or —I(W)(BF₄), and W is as hereinbefore defined (and, where the compound of formula III contains more than one W group, they are preferably all the same), for example in the presence of a catalyst containing, preferably, Pd or Cu, and a base, such as potassium or sodium hydroxide, potassium carbonate, potassium tert-butoxide and lithium N,N-diisopropylamide. Catalysts that may be mentioned include Pd₂(dba)₃ (tris(dibenzylideneacetone)-dipalladium(0)), bases that may be mentioned include cesium carbonate, ligands that may be mentioned include 2,2-bis(diphenylphosphino)-1,1′-binaphthyl and solvents that may be employed include toluene. Such reactions may be performed at elevated temperature (e.g. at about 90° C.) under an inert (e.g. argon) atmosphere.
(iii) Reaction of a compound of formula IV,

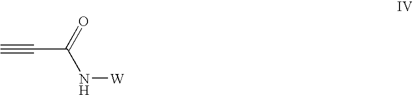

wherein W is as hereinbefore defined, or a N-protected derivative thereof, with a suitable reagent that provides a source of azide ions, such as sodium azide or trimethylsilyl azide, under conditions known to those skilled in the art. The reaction may be performed under standard 1,3-dipolar cycloaddition reaction conditions, such as those described in Katritzky A. R. et al., Heterocycles 2003, 60 (5), 1225-1239. For example, the reaction may be performed without solvent or in the presence of an appropriate solvent (e.g. water, methanol, ethanol, dimethylformamide, dichloromethane, tetrahydrofuran, dioxane, toluene or mixtures thereof) at about room temperature or above (e.g. between 40 and 80° C.).
(iv) Reaction of triazole, or a protected derivative thereof, with an appropriate base (or a mixtures of bases), such as potassium bis(trimethylsilyl)amide, sodium bis(trimethylsilyl)amide, sodium hydride, potassium tert-butoxide or an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)) followed by reaction with a compound of formula V,

W—N═C═O V

wherein W is as hereinbefore defined, followed by quenching with a suitable proton source (e.g. water or aqueous, saturated NH₄Cl solution). The skilled person will appreciate that the triazole may need to be protected at the nitrogen atom of the triazole ring system, preferably with a protective group that is also a directing metallation group (such as a SEM (i.e. a —CH₂OC₂H₄Si(CH₃)₃) group). The reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere followed (as appropriate) by deprotection of the N-protective group under standard conditions (e.g. in the case of the SEM group, employing conditions such as the presence of HCl in ethanol).
(v) Reaction of a compound of formula VI,

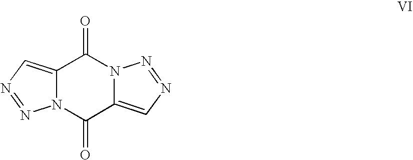

with a compound of formula II as hereinbefore defined, for example under coupling conditions such as those described hereinbefore in respect of process step (i) above. Preferred conditions include reaction in the presence of base, solvent but no coupling reagent. In this case, the compound of formula II may also be employed in excess.

1,2,3-Triazole-4-carboxylic acid is commercially available (e.g. from Pfaltz & Bauer Chemicals), or may be prepared from propiolic acid and a source of azide ions, for example employing reagents and under conditions such as those described hereinbefore in respect of preparation of compounds of formula I (process step (iii)).

Compounds of formula II may be prepared:

(I) by reaction of a compound of formula III, as hereinbefore defined, with ammonia, or preferably with a protected derivative thereof (e.g. benzylamine), under conditions such as those described hereinbefore in respect of preparation of compounds of formula I (process step (ii)); or
(II) by reduction of a compound of formula VII,

W—NO₂ VI;

wherein W is as hereinbefore defined, under standard reduction conditions, for example, by employing tin (II) chloride dehydrate in the presence of an alcoholic solvent (e.g. ethanol) at reflux or by hydrogenation in the presence of a catalyst (e.g. palladium on carbon), with a source of hydrogen (e.g. hydrogen gas or nascent hydrogen (e.g. from ammonium formate)), optionally in the presence of a solvent (such as an alcoholic solvent (e.g. methanol)).

1,2,3-Triazole-4-carboxylic acid amide may be prepared by reaction of 1,2,3-triazole-carboxylic acid, or a derivative thereof, with ammonia, for example under reaction conditions such as those described hereinbefore in respect of preparation of compounds of formula I (process step (i) above).

Compounds of formula IV may be prepared by reaction of propiolic acid with a compound of formula II as hereinbefore defined, for example under reaction conditions such as those described hereinbefore in respect of preparation of compounds of formula I (process step (i) above).

Compounds of formula VI may be prepared from 1,2,3-triazole-4-carboxylic acid under dimerising conditions, for example in the presence of thionyl chloride or oxalyl chloride (optionally in the presence of a suitable solvent and catalyst, such as one hereinbefore defined in respect of process step (i)). Other dimerising reagents include carbodiimides, such as 1,3-dicyclohexylcarbodiimide or 1-(3 dimethylaminopropyl)-3-ethylcarbodiimide (EDCI, or hydrochloride thereof) optionally in the presence of a suitable base (e.g. 4-dimethylaminopyridine).

Compounds of formulae III, V and VII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

Substituents on W (if present) as hereinbefore defined may be modified one or more times, after or during the processes described above for preparation of compounds of formula I by way of methods that are well known to those skilled in the art. Examples of such methods include substitutions, reductions, oxidations, alkylations, acylations, hydrolyses, esterifications and etherifications. The precursor groups can be changed to a different such group, or to the groups defined in formula I, at any time during the reaction sequence. In the case where the substituent on W represents a halo group, such groups may be inter-converted one or more times, after or during the processes described above for the preparation of compounds of formula I. Appropriate reagents include NiCl₂ (for the conversion to a chloro group). In this respect, the skilled person may also refer to “Comprehensive Organic Functional Group Transformations” by A. R. Katritzky, O. Meth-Cohn and C. W. Rees, Pergamon Press, 1995.

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of the invention may be isolated from their reaction mixtures using conventional techniques.

It will be appreciated by those skilled in the art that, in the processes described above and hereinafter, the functional groups of intermediate compounds may need to be protected by protecting groups. For example the triazole nitrogen or (when there is an —N(R^{4b})R^{5b} substituent on W) the nitrogen of the —N(R^{4b})R^{5b} group may need to be protected. Suitable nitrogen-protecting groups include those which form:

(i) carbamate groups (i.e. alkoxy- or aryloxy-carbonyl groups);
(ii) amide groups (e.g. acetyl groups);
(iii) N-alkyl groups (benzyl or SEM groups);
(iv) N-sulfonyl groups (e.g. N-arylsulfonyl groups);
(v) N-phosphinyl and N-phosphoryl groups (e.g. diarylphosphinyl and diarylphosphoryl groups); or
(vi) N-silyl group (e.g. a N-trimethylsilyl group).

Further protecting groups for the triazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Orgasmic Synthesis”, 3^{rd} edition, T. W. Greene & P.G.M. Wutz, Wiley-Interscience (1999).

### Medical and Pharmaceutical Uses

Compounds of the invention are useful because they possess pharmacological activity. Such compounds are therefore indicated as pharmaceuticals. According to a further aspect of the invention there is provided a compound of formula I, as hereinbefore defined, or a pharmaceutically-acceptable salt thereof, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of the invention. All prodrugs of compounds of the invention are included within the scope of the invention.

By “prodrug of a compound of the invention”, we include compounds that form a compound of the invention, in an experimentally-detectable amount, within a predetermined time (e.g. about 1 hour), following oral or parenteral administration.

Compounds of the invention are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of the invention are thus expected to be useful in the treatment of inflammation.

The term “inflammation” will be understood by those skilled in the art to include any condition characterised by a localised or a systemic protective response, which may be elicited by physical trauma, infections chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of the invention may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of formula I, as hereinbefore defined but without the provisos or a pharmaceutically-acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I, as hereinbefore defined, or a pharmaceutically-acceptable salt thereof, in admixture with a pharmaceutically acceptable adjuvant, diluent or carrier.

The invention further provides a process for the preparation of a pharmaceutical formulation, as hereinbefore defined, which process comprises bringing into association a compound of formula I, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:

(A) a compound of formula I, as hereinbefore defined but without the provisos, or a pharmaceutically-acceptable salt thereof, and
(B) another therapeutic agent that is useful in the treatment of inflammation, wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of the invention and at least one comprises the other therapeutic agent, or may be presented (i.e. formulated) as a combined preparation (i.e. presented as a single formulation including compound of the invention and the other therapeutic agent).

Thus, there is further provided:

(1) a pharmaceutical formulation including a compound of formula I, as hereinbefore defined but without the provisos, or a pharmaceutically-acceptable salt thereof, another therapeutic agent that is useful in the treatment of inflammation, and a pharmaceutically-acceptable adjuvant, diluent or carrier; and
(2) a kit of parts comprising components:
(a) a pharmaceutical formulation including a compound of formula I, as hereinbefore defined but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier, which components (a) and (b) are each provided in a form that is suitable for administration in conjunction with the other.

The invention further provides a process for the preparation of a combination product as hereinbefore defined, which process comprises bringing into association a compound of formula I, as hereinbefore defined but without the provisos, or a pharmaceutically acceptable salt thereof with the other therapeutic agent that is useful in the treatment of inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective and/or selective inhibitors of lipoxygenases, and particularly 15-lipoxygenase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior ark whether for use in the stated indications or otherwise.

### Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (PBS) (pH 7.4);
b) inhibitor (i.e. compound) or vehicle (0.5 μl DMSO);
c) 10 μL of a 10× concentrated solution of 15-lipoxygenase in PBS. The plates are incubated for 5 minutes at room temperature;

d) 5 μl of 0.125 mM arachidonic acid in PBS. The plate is then incubated for 10 minutes at room temperature;

e) the enzymatic reaction is terminated by the addition of 100 μl methanol; and
f) the amount of 15-hydroperoxy-eicosatetraenoic acid or 15-hydroxy-eicosatetraenoic acid is measured by reverse phase HPLC.

The invention is illustrated by way of the following examples, in which the following abbreviations may be employed:
aq. aqueous
DMAP 4-dimethylaminopyridine
DMF dimethylformamide
DMSO dimethylsulfoxide
EtOAc ethyl acetate
MS mass spectrum
NMR nuclear magnetic resonance
Pd—C palladium on activated carbon
PyBrop Bromotripyrrolidinophosphonium hexafluorophosphate
rt room temperature
TBTU O-benzotriazol-1-yl-N,N,N′,N′-tetramethyluronium tetrafluoroborate
THF tetrahydrofuran

Starting materials and chemical reagents specified in the synthesis described below are commercially available from, e.g. Sigma-Aldrich Fine Chemicals.

Unless otherwise stated, one or more tautomeric forms of compounds of the examples described hereinafter may be prepared in situ and/or isolated ALI tautomeric forms of compounds of the examples described hereinafter should be considered to be disclosed.

### Synthesis of Intermediates

### 1,2,3-Triazole-4-carboxylic Acid

A mixture of propiolic acid (1.55 mL, 1.76 g, 25 mmol), azidotrimethylsilane (8.4 mL, 7.3 g, 63 mmol) and MeOH (10 mL) was stirred at 80° C. for 3 h in a sealed vial. After cooling to rt the white solid formed was filtered off, washed with Et₂O (2×50 mL) and dried. Yield 2.11 g (74%).

¹H NMR (DMSO-d₆, 400 MHz) δ 13.30 (br. s, 2H), 8.40 (s, 1H).

### 1-[2-(Trimethylsilyl)ethoxymethyl]-1,2,3-triazole

NaH (60% suspension in mineral oil, 1.10 g, 28.4 mmol) was added to a solution of 1,2,3-triazole (1.90 g, 27.0 mmol) in THF (30 mL) and the mixture was stirred at rt for 1 h. The mixture was cooled in an ice bath and 2-(trimethylsilyl)-ethoxymethyl chloride (5.0 g, 30 mmol) was added dropwise. The mixture was allowed to warm to rt and stirred at rt for 18 h. The precipitate was filtered off and the filtrate was concentrated and redissolved in Et₂O (50 mL). The solution was washed with water (20 mL), dried (Na₂SO₄) and concentrated to give a colourless oil (5.7 g). According to the ¹H NMR spectrum, the oil was a mixture (3:1) of the title product and the isomeric 2-[2-trimethylsilyl)ethoxymethyl]-1,2,3-thiazole. The mixture was used without further purification.

¹H NMR (CDCl₃, 400 MHz) δ 7.76-7.73 (m, 2H), 5.71 (s, 2H), 3.54 (t, 2H), 0.94 (t, 2H), −0.02 (s, 9H).

### Synthesis of Arylamine Intermediates

Arylamines which were not commercially available were synthesised in accordance with procedures known to those skilled in the art, for example, such as those described hereinafter.

### 2-Aminoquinoxaline

### (a) 2-Benzylaminoquinoxaline

A mixture of 2-chloroquinoxaline (1.10 g, 6.68 mmol) and benzylamine (6 mL) was heated at 150° C. for 6 h. After cooling to rt the mixture was poured into NaH₂PO₄ (aq, sat, 50 mL) and extracted with EtOAc (3×20 mL). The combined extracts were dried (Na₂SO₄), concentrated and purified by chromatography to give the sub-title compound (1.35 g, 87%) as a yellow oil.

¹H NMR (DMSO-d₆, 400 MHz) δ 8.37 (s, 1H), 8.10 (t, 1H), 7.76 (d, 1H), 7.54-7.25 (m, 7H), 4.63 (d, 2H).

### (b) 2-Aminoquinoxaline

A mixture of 2-benzylaminoquinoxaline (1.30 g, 5.50 mmol), ammonium formate (3.13 g, 49.7 mmol) and Pd—C (10% Pd, 130 mg) in MeOH (60 ml) was stirred at rt for 48 h. The mixture was filtered through Celite®, concentrated and purified by chromatography to give the title compound (278 mg, 25%) as an orange oil.

¹H NMR (DMSO-d₆, 400 MHz) δ 8.26 (s, 1H), 7.74 (d, 1H), 7.55-7.46 (m, 2H), 7.30 (ddd, 1H), 6.95 (s, 2H).

### 4-Chloro-o-anisidine

A mixture of 4-chloro-2-methoxy-1-nitrobenzene (938 mg, 5.0 mmol), tin(II) chloride dihydrate (3.38 g, 15 mmol) and EtOH (25 mL) was heated at reflux for 18 h. After cooling to rt, NaOH (aq, 4M, 50 mL) was added. The mixture was extracted with Et₂O (3×20 mL) and the combined extracts dried (Na₂SO₄) and concentrated. Purification by chromatography gave the title compound (511 mg, 65%) as a red oil which solidified on standing.

¹H NMR (DMSO-d₆, 400 MHz) δ 6.78 (1H, d), 6.67 (1H, dd), 6.57 (1H, d), 4.82 (2H, s), 3.75 (3H, s).

### 2,4-Dichloro-m-toluidine

This intermediate was prepared in accordance with the procedure described above from 1,3-dichloro-2-methyl-4-nitrobenzene (1.03 g, 5 mmol) to provide an off-red oil which solidified on standing. Yield 617 mg (70%).

¹H NMR (DMSO-d₆, 400 MHz) δ 7.05 (1H, d), 6.64 (1H, d), 5.44 (2H, s), 2.32 (3H, s).

### 4-Amino-N,N-diethylbenzenesulfonamide

A solution of diethylamine (5.2 g, 710 mmol) in pyridine (15 mL) was cooled in an ice bath and N-acetylsulfanilyl chloride (10 g, 43 mmol) was added in small portions during 10 min. The mixture was stirred at 110° C. for 4 h and concentrated to give a brown oil. EtOH (15 mL), water (25 mL) and HCl (aq, conc, 25 mL) were added and the mixture was stirred at 100° C. for 3 h. After cooling to r, the pH was adjusted to ˜10 by the addition of NaOH (aq, 40%). The brown precipitate was filtered off, washed with water, dried and recrystallised from Et₂O/heptane to give the title product (6.0 g, 62%) as yellow crystals.

¹H NMR (DMSO-d₆, 400 MHz) δ 7.39 (dd, 2H), 6.61 (dd, 2H), 5.94 (s, 2H), 3.05 (q, 4H), 1.01 (t, 6H).

### 4-Amino-N-methylbenzenesulfonamide

### (a) N-Methyl-4-nitrobenzenesulfonamide

A mixture of 4-nitrobenzenesulfonyl chloride (1.20 g, 5.42 mmol), methylamine (2M in THF, 2.7 mL, 5.4 mmol), DMAP (66 mg, 0.54 mmol), triethylamine (0.87 nm, 6.23 mmol) and CH₂Cl₂ (50 mL) was stirred at rt for 15 ml. The mixture was diluted with CH₂Cl₂ (100 mL), washed with HCl (aq, 1M, 50 mL) and NaCl (aq, sat, 50 mL), dried Na₂SO₄) and concentrated. Purification by chromatography (eluent EtOAc/heptane) gave the sub-title compound (337 mg, 29%) as light yellow needles.

¹H NMR (DMSO-d, 400 M) δ 8.41 (2H, ddd), 8.01 (211, ddd), 7.95-7.76 (1H, br. s), 2.47 (3H, s).

### (b) 4-Amino-N-methylbenzenesulfonamide

A mixture of N-methyl-4-nitrobenzenesulfonamide (337 mg, 1.56 mmol), Pd—C (10% Pd, 100 mg) and a few drops of DMF in MeOH (20 mL) was hydrogenated at normal pressure and temperature for 3 days. The mixture was filtered through Celite® and concentrated to give the title product (207 mg, 71%) as brown crystals.

¹H NMR (DMSO-d₆, 400 MHz) δ 7.40 (2H, ddd), 6.90 (1H, q), 6.61 (2H, ddd), 5.91 (2H, s), 2.32 (3H, d).

### 4-Amino-N,N-dimethylbenzenesulfonamide

### (a) N,N-Dimethyl-4-nitrobenzenesulfonamide

The sub-title compound was prepared from 4-nitrobenzenesulfonyl chloride (120 g, 5.42 mmol) and dimethylamine hydrochloride (508 mg, 6.23 mmol) using an excess of triethylamine (1.73 mL, 12.45 mmol) in accordance with the procedure described above. Yield 818 mg (66%) as off-yellow needles.

¹H NMR (DMSO-d₆, 400 MHz) δ 8.43 (2H, ddd), 8.00 (2H, ddd), 2.67 (6H, s).

### (b) 4-Amino-N,N-dimethylbenzenesulfonamide

The title compound was prepared from N,N-dimethyl-4-nitrobenzenesulfonamide (767 mg, 3.33 mmol) by hydrogenation in accordance with the procedure described hereinbefore. Yield 608 mg (91%) as a brown solid.

¹H NMR (DMSO-d₆, 400 MHz) δ 7.33 (2H, ddd), 6.62 (2H, ddd), 6.2-5.8 (2H, br. s), 2.48 (6H, s).

3-Amino-6-fluoroquinoline, 3-amino-7-fluoroquinoline, 3-amino-8-fluoroquinoline and 3-amino-8-chloroquinoline were prepared in accordance with the steps (a) to (f) described below.

### (a) 2-[(4-Fluorophenylamino)methylene]malonic Acid Diethyl Ester

A mixture of 4-fluoroaniline (4.26 mL, 45 mmol) and 2-ethoxymethylenemalonic acid diethyl ester (14.59 g, 67.5 mmol) was stirred at 130° C. for 18 h. After cooling to rt, the solid was recrystallised from acetone/water to give the sub-title compound (9.84 g, 78%) as a shiny off-white solid.

¹H NMR (DMSO-d₆, 400 MHz) δ 10.67 (1H, s), 8.31 (1H, s), 7.45-7.39 (2H, m), 7.21 (2H, t), 4.25-4.05 (4H, m), 1.25 (6H, t).

### 2-[(3-Fluorophenylamino)methylene]malonic Acid Diethyl Ester

The sub-title compound was prepared from 3-fluoroaniline (1.83 g, 16.5 mmol) in accordance with the procedure described above, except that the crude product was used without purification.

¹H NMR (DMSO-d₆, 400 MHz) δ 10.66 (1H, d), 8.38 (1H, d), 7.46-7.33 (2H, m), 7.21 (1H, dd), 6.97 (1H, dt), 4.20-4.05 (4H, m), 1.3-1.2 (6H, m).

### 2-[(2-Fluorophenylamino)methylene]malonic Acid Diethyl Ester

The sub-title compound was prepared from 2-fluoroaniline (5.0 g, 45 mmol) in is accordance with the procedure described above. Yield 11.68 g (92%) as a white cotton-like solid.

¹H NMR (DMSO-d₆, 400 MHz) δ 11.05 (1H, d), 8.62 (1H, d), 7.79 (1H, dt), 7.49 (1H, ddd), 7.40 (1H, ddd), 7.33 (1H, ddd), 4.37 (2H, q), 4.28 (2H, q), 1.42 (3H, t), 1.41 (3H, t).

### 2-[(2-Chlorophenylamino)methylene]malonic Acid Diethyl Ester

The sub-title compound was prepared from 2-chloroaniline (4.74 mL, 45 mmol) in accordance with the procedure described above. Yield 12.66 g (94%) as a white solid.

¹H NMR (DMSO-d₆, 400 MHz) δ 11.17 (1H, d), 8.51 (1H, d), 7.65 (1H, d), 7.55 (1H, d), 7.40 (1H, dd), 7.16 (1H, dd), 4.23 (2H, q), 4.14 (2H, q), 1.27 (3H, t), 1.26 (3H, t).

### (b) 6-Fluoro-4-hydroxyquinoline-3-carboxylic Acid Ethyl Ester

2-[(6-Fluorophenylamino)methylene]malonic acid diethyl ester (9.83 g, 34.9 mmol; see step (a) above) was added to Dowtherm® A (5 mL). The mixture was heated to 220° C. and kept at that temperature for 1.5 h. After cooling to rt, the precipitate was filtered off, washed with EtOAc/heptane (2:1) and dried. Yield 4.15 g (51%) as a white solid.

¹H NMR (DMSO-d₆, 400 M) 12.43 (1H, s), 8.56 (1H, s), 7.80-7.58 (3H, m), 4.20 (2H, q), 1.28 (3H, t).

### 7-Fluoro-4-hydroxyquinoline-3-carboxylic Acid Ethyl Ester

The sub-title compound was prepared from crude 2-[(3-fluorophenylamino)-methylene]malonic acid diethyl ester (see step (a) above) in accordance with the procedure described above. Yield 2.46 g (66% for two steps) as an off-white solid.

¹H NMR (DMSO-d₆, 400 MHz) δ 12.32 (1H, s), 8.60 (1H, s), 8.14 (1H, d), 7.67 (1H, dd), 7.45 (1H, dd), 4.22 (2H, q), 1.28 (3H, t).

### 8-Fluoro-4-hydroxyquinoline-3-carboxylic Acid Ethyl Ester

The sub-title compound was prepared from 2-[(2-fluorophenylamino)-methylene]malonic acid diethyl ester (11.67 g, 41.4 mmol; see step (a) above) in accordance with the procedure described above. Yield 6.11 g (63%) as a white solid.

¹H NMR (DMSO-d₆, 400 MHz) δ 12.45 (1H, s), 8.37 (1H, s), 7.94 (1H, d), 7.64 (1H, ddd), 7.39 (1H, ddd), 4.22 (2H, q), 1.28 (3H, t).

### 8-Chloro-4-hydroxyquinoline-3-carboxylic Acid Ethyl Ester

The sub-title compound was prepared from 2-[(2-chlorophenylamino)-methylene]malonic acid diethyl ester (12.64 g, 42.5 mmol; see step (a) above) in accordance with the procedure described above. Yield 7.94 g (74%) as a white solid.

¹H NMR (DMSO-d₆, 400 M 5) δ 11.89 (1H, s), 8.41 (1H, s), 8.11 (1H, dd), 7.88 (1H, dd), 7.41 (1H, t), 4.22 (2H, q), 1.28 (3H, t).

### (c) 4-Chloro-6-fluoroquinoline-3-carboxylic Acid Ethyl Ester

A mixture of 6-fluorohydroxyquinoline-3-carboxylic acid ethyl ester (4.15 g, 17.6 mmol; see step (b) above) and POCl₃ (5.40 g, 35.2 mmol) was stirred at 100° C. for 30 min. After cooling to rt, the mixture was poured onto ice (˜50 g) and neutralised with ammonia (aq, sat, 20 mL). The mixture was extracted with CH₂Cl₂ (3×30 mL) and the combined extracts washed with ammonia (aq, 2M, 20 mL) and concentrated to give the sub-title compound (4.29 g, quantitative yield) as shiny flakes.

¹H NMR (DMSO-d₆, 400 MHz) δ 9.22 (1H, s), 8.33 (1H, dd), 8.16 (1H, dd), 8.02 (1, ddd), 4.54 (2H, q), 1.50 (3H, t).

### 4-Chloro-7-fluoroquinoline-3-carboxylic Acid Ethyl Ester

A mixture of 7-fluoro-4-hydroxyquinoline-3-carboxylic acid ethyl ester (2.45 g, 10.0 mmol; see step (b) above) and POCl₃ (3 mL) was stirred at 100° C. for 20 nm in, cooled and concentrated. The residue was washed with heptane (3×30 mL) and dried. Yield 2.26 g (89%) as an off-white solid.

¹H-NMR (CDCl₃, 400 M) δ 9.52 (1H, s), 8.73 (1H, dd), 8.32 (1H, dd), 7.82 (1H, ddd), 4.57 (2H, q), 1.51 (3H, t).

### 4-Chloro-8-fluoroquinoline-3-carboxylic Acid Ethyl Ester

A mixture of 8-fluoro-4-hydroxyquinoline-3-carboxylic acid ethyl ester (6.11 g, 26.0 mmol; see step (b) above) and POCl₃ (20 mL) was stirred at 100° C. for 3.5 h. cooled and concentrated to give a yellow semi-solid (9.85 g) which was used without purification.

¹H-NMR (CDCl₃, 400 MHz) δ 9.58 (1H, s), 8.46 (1H, d), 8.04-7.90 (2H, m), 4.60 (2H, q), 1.54 (31, t).

### 4,8-Dichloroquinoline-3-carboxylic Acid Ethyl Ester

A mixture of 8-chloro-4-hydroxyquinoline-3-carboxylic acid ethyl ester (7.94 g, 31.5 mmol; see step (b) above) and POCl₃ (6 mL) was stirred at 100° C. for 30 min, cooled and concentrated. The crude material was recrystallised from EtOAc. Yield 5.46 g (68%) as white flakes.

¹H-NMR (CDCl₃, 400 MHz) δ 9.23 (1H, s), 8.34 (1H, dd), 8.16 (1H, dd), 7.81 (1H, dd), 4.44 (2H, q), 1.39 (3H, t).

### (d) 6-Fluoroquinoline-3-carboxylic Acid Ethyl Ester

A mix of 4-chloro-6-fluoroquinoline-3-carboxylic acid ethyl ester (4.2 g, 17.6 mmol; see step (c) above) and Pd—C (10% Pd, 100 mg) in acetic acid (10 mL) was hydrogenated at normal pressure and temperature for 18 h. The mixture was filtered through Celite® which was additionally washed with EtOAc (30 mL). The combined filtrates were concentrated and the residue recrystallised from EtOAc/heptane to give the sub-title compound (931 mg, 24%) as a light orange solid.

¹H NMR (DMSO-d₆, 400 MHz) δ 9.28 (1H, s), 9.02 (1H, s), 8.18 (1H, dd), 8.06 (1H, dd), 7.84 (1H, m), 4.42 (2H, q), 1.39 (3H, t).

### 7-Fluoroquinoline-3-carboxylic Acid Ethyl Ester

The sub-title compound was prepared from 4-chloro-7-fluoroquinoline-3-carboxylic acid ethyl ester (1.50 g, 5.91 mmol; see step (c) above) in accordance with the procedure described above. The green crude product was used without purification.

¹H NMR (DMSO-d₆, 400 MHz) δ 9.33 (1H, d), 9.07 (1H, dd), 8.36 (1H, dd), 7.88 (1H, dd), 7.69 (1H, dt), 4.42 (2H, q), 1.39 (3H, t).

### 8-Fluoroquinoline-3-carboxylic Acid Ethyl Ester

The sub-title compound was prepared from 4-chloro-8-fluoroquinoline-3-carboxylic acid ethyl ester (9.65 g of the crude material; see step (c) above) by hydrogenation for 48 h in accordance with the procedure described above. The brown oil obtained was used without purification.

¹H NMR (DMSO-d₆, 400 MHz) δ 9.33 (1H, d), 9.07 (1H, dd), 8.06 (1H, dd), 7.78-7.65 (2H, m), 4.42 (2H, q), 1.39 (3H, t).

### 8-Chloroquinoline-3-carboxylic Acid Ethyl Ester

The sub-title compound was prepared from 4,8-dichloroquinoline-3-carboxylic acid ethyl ester (5.15 g, 20.1 mmol; see step (c) above) by hydrogenation for 2 h in accordance with the procedure described above. The product was purified by chromatography (eluent EtOAc/heptane). Yield 717 mg (15%) of a white solid.

¹H NMR (DMSO-d₆, 400 MHz) δ 9.41 (1H, d), 9.09 (1H, d), 8.23 (1H, dd), 8.12 (1H, dd), 7.71 (1H, t), 4.44 (2H, q), 1.40 (3H, t).

### (e) 6-Fluoroquinoline-3-carboxylic Acid

NaOH (aq, 2M, 8 mL, 16 mmol) was added to a mixture of 6-fluoroquinoline-3-carboxylic acid ethyl ester (927 mg, 4.23 mmol; see step (d) above), MeOH (15 mL) and dioxane (10 mL). The mixture was stirred at rt for 30 min, acidified with HCl (2M, 12 mL) and extracted with EtOAc (3×20 mL). The combined extracts were dried (Na₂SO₄) and concentrated to give the title compound (600 mg, 74%) as a light yellow solid.

¹H NMR (DMSO-d₆, 400 MHz) δ 9.26 (1H, d), 8.96 (1H, d), 8.15 (1H, dd), 8.01 (1H, dd), 7.81 (1H, ddd).

### 7-Fluoroquinoline-3-carboxylic Acid

The sub-title compound was prepared from 7-fluoroquinoline-3-carboxylic acid ethyl ester (crude material; see step (d) above) in accordance with the procedure described above. Yield 176 mg (16% over two steps) as a white solid.

¹H NMR (DMSO-d₆, 400 z), 13.83-13.26 (1H, br. s), 9.33 (1H, d), 9.03 (1H, d), 8.33 (1H, dd), 7.86 (1H, dd), 7.67 (1H, dt).

### 8-Fluoroquinoline-3-carboxylic Acid

The sub-title compound was prepared from 8-fluoroquinoline-3-carboxylic acid ethyl ester (9.6 g of the crude material; see step (d) above) in accordance with the procedure described above. Yield 3.00 g (60% over three steps) as a light yellow solid.

¹H NMR (DMSO-d₆, 400 MHz) δ 9.30 (1H, d), 9.01 (1H, dd), 8.00 (1H, dd), 7.74-7.62 (2H, m).

### 8-Chloroquinoline-3-carboxylic Acid

The sub-title compound was prepared from 8-chloroquinoline-3-carboxylic acid ethyl ester (712 mg, 3.02 mmol; see step (d) above) in accordance with the procedure described above. Yield 495 mg (79%) as a white solid.

¹H NMR (DMSO-d₆, 400 MHz) δ 13.7 (1H, s), 9.40 (1H, d), 9.06 (1H, d), 8.22 (1H, dd), 8.10 (1H, dd), 7.69 (1H t).

### (f) 3-Amino-6-fluoroquinoline

A mixture of 6-fluoroquinoline-3-carboxylic acid (595 mg, 3.11 mmol; see step (e) above), diphenylphosphoryl azide (991 mg, 3.6 mmol), triethylamine (364 mg, 3.6 mmol) and anhydrous THF (15 mL) was heated at reflux for 2 h Water (5 mL) was added and the mixture was heated at reflux for 2 h. After cooling to rt, the mixture was extracted with EtOAc (3×15 mL) and the combined extracts dried Na₂SO₄) and concentrated. The residue was recrystallised from toluene to give title compound (142 mg, 28%) as a yellow solid.

¹H NMR (DMSO-d₆, 400 MHz) δ 8.40 (1H, d), 7.79 (1H, dd), 7.38 (1H, dd), 7.17 (1H, dt), 7.09 (1H, d), 5.82 (2H, s).

### 3-Amino-7-fluoroquinoline

The sub-title compound was prepared from 7-fluoroquinoline-3-carboxylic acid (172 mg, 0.90 mmol; see step (e) above) in accordance with the procedure described above. Yield 43 mg (29%) as a yellow solid.

¹H NMR (DMSO-d₆, 400 z) δ 8.46 (1H, d), 7.69 (1H, dd), 7.49 (1H, dd), 7.31 (1H, dd), 7.19 (1H, d), 5.63 (2H, s).

### 3-Amino-8-fluoroquinoline

The sub-title compound was prepared from 8-fluoroquinoline-3-carboxylic acid (1.00 g, 5.23 mmol; see step (e) above) in accordance with the procedure described above. Yield 113 mg (13%) as a yellow solid.

¹H-NMR (DMSO-d₆, 400 MHz) δ 8.42 (1H, d), 7.38 (1H, dd), 7.29 (1H, ddd), 7.13 (1H, dd), 7.05 (1H, dd), 5.85 (2H, s).

### 3-Amino-8-chloroquinoline

The sub-title compound was prepared from 8-chloroquinoline-3-carboxylic acid (491 mg, 2.37 mmol; see step (e) above) in accordance with the procedure described above. Yield 169 mg (40%) as a yellow solid.

¹H NMR (DMSO-d₆, 400 MHz) δ 8.53 (1H, d), 7.59 (1H, dd), 7.46 (1H, dd), 7.33 (1H, t), 7.18 (1H, dd), 5.89 (2H, s).

### 2-Amino-5,6-dimethoxypyridine

### (a) 2-Bromo-3-methoxy-6-nitropyridine

2-Bromo-3-methoxypyridine (4.45 g, 23.7 mmol) was added to a mixture of fuming HNO₃ and concentrated H₂SO₄ (1:1, 18 mL) at 0° C. The mixture was stirred at 55° C. for 1.5 h and then poured into ice water (150 mL). The precipitate formed was filtered off, washed with water (3×100 mL) and dried in vacuo to give 3.54 g (64%) of slightly yellow solid, which was essentially pure product.

¹H NMR (DMSO-d₆, 400 MHz) δ 8.41 (d, 1H), 7.80 (d, 1H), 4.06 (s, 3H).

### (b) 2,3-Dimethoxy-6-nitropyridine

Sodium methoxide (927 μL of 30% solution in MeOH, 5.2 mmol) was added to a mixture of 2-bromo-3-methoxy-6-nitropyridine (750 mg, 3.22 mmol), DMSO (6 mL) and MeOH (9 mL). The mixture was stirred at rt for 90 min, then at 35° C. for 24 h and at rt for 24 h. The mixture was poured into ice water (150 mL) and the precipitate filtered off, washed with water (100 mL) and dried in vacuo to provide 453 mg (76%) of the sub-title compound as a slightly yellow solid.

¹H NMR (DMSO-d₆, 400 MHz) δ 8.02 (d, 1H), 7.55 (d, 1H), 3.97 (s, 3H), 3.94 (s, 3H).

### (c) 2-Amino-5,6-dimethoxypyridine

A mixture of 2,3-dimethoxy-6-nitropyridine (450 mg, 2.44 mmol), Pd—C (10%, 100 mg), MeOH (10 mL) and CH₂Cl₂ (10 mL) was hydrogenated at ambient temperature and pressure for 3 h. The mixture was filtered through Celite® and the filtrate concentrated in vacuo to give the title product (356 mg, 95%) as a light brown solid.

¹H NMR (DMSO-d₆, 400 MHz) δ 7.05 (d, 1H), 5.92 (d, 1H), 5.36 (br. s, 2H), 3.75 (s, 3H), 3.60 (s, 3H).

### 2-Amino-5-methoxypyridine

A mixture of 2-bromo-3-methoxy-6-nitropyridine (1.20 g, 5.15 mmol), hydrazine hydrate (6 mL) and Pd—C (10%, 400 mg) in EtOH (40 mL) was heated at reflux for 45 min. The mixture was filtered through Celite® and concentrated in vacuo. Water (20 mL) and NH₃ (aq., sat.; 10 mL) were added and the mixture was extracted with CHCl₃ (2×50 mL). The combined extracts were dried (Na₂SO₄) and concentrated in vacuo to give the title product (615 mg, 96%) as a low melting colourless solid.

¹H NMR (DMSO-d₆, 400 MHz) δ 7.64 (dd, 1 μl), 7.10 (dd, 1H), 6.42 (dd, 1H), 5.43 (br. s, 2H), 3.68 (s, 3H).

### 2-Amino-3-methoxypyridine

Prepared by a procedure analogous to that described above for 2-amino-5,6-dimethoxypyridine, step (c), using 3-methoxy-2-nitropyridine (1.598 g, 10.4 mmol) in place of 2,3-dimethoxy-6-nitropyridine. Yield: 961 mg (74%) of white needles.

¹H NMR (DMSO-d₆, 400 MHz) δ 7.49 (dd, 1H), 6.99 (dd, 1H), 6.49 (dd, 1H), 5.60 (br. s, 2H), 3.76 (s, 3H).

### 2-Amino-5-ethoxypyridine

### (a) 2-Bromo-3-ethoxypyridine

A mixture of 2-bromopyridin-3-ol (2.00 g, 11.5 mmol), iodoethane (3.12 g, 20 mmol) and K₂CO₃ (2.49 g, 18 mmol) in DMF (17 mL) was stirred at 80° C. for 110 min. The mixture was concentrated in vacuo and the residue partitioned between EtOAc (100 mL) and water (50 mL). The aqueous phase was extracted with EtOAc (50 mL), the combined organic phases washed with water (25 mL) and NaCl (aq., sat; 25 mL), dried (Na₂SO₄) and concentrated in vacuo to give the sub-title compound (2.15 g, 92%) as a brown oil.

¹H NMR (DMSO-d₆, 400 MHz) δ 7.95 (dd, 1H), 7.51 (dd, 1H), 7.39 (dd, 1H), 4.15 (q, 2H), 1.36 (t, 3H).

### (b) 2-Bromo-3-ethoxy-6-nitropyridine

Prepared by a procedure analogous to that described above for 2-bromo-3-methoxy-6-nitropyridine using 2-bromo-3-ethoxypyridine (1.827 g, 9.04 mmol) in place of 2-bromo-3-methoxypyridine. Yield: 1.53 g (68%) of slightly yellow solid.

¹H NMR (DMSO-d₆, 400 MHz) δ 8.39 (d, 1H), 7.79 (d, 1H), 4.33 (q, 2H), 1.42 (t, 3H).

### (c) 2-Amino-5-ethoxypyridine

Prepared by a procedure analogous to that described above for 2-amino-5-methoxypyridine using 2-bromo-3-ethoxy-6-nitropyridine (1.50 g, 6.08 mmol) in place of 2-bromo-3-methoxy-6-nitropyridine. Yield: 836 mg (100%) of yellow oil.

¹H NMR (DMSO-d₆, 400 MHz) δ 7.62 (d, 1H), 7.09 (dd, 1H), 6.40 (d, 1H), 5.42 (br. s, 2H), 3.91 (q, 2H), 1.26 (t, 3H).

### 2-Amino-5-propoxypyridine

### (a) 2-Bromo-3-propoxypyridine

Prepared by a procedure analogous to that described above for 2-bromo-3-ethoxypyridine using 1-iodopropane in place of iodoethane. Yield: 2.26 g (91%) of light-brown oil.

¹H NMR (DMSO-d₆, 400 MHz) δ 7.95 (dd, 1H), 7.51 (dd, 1H), 7.39 (dd, 1H), 4.06 (t, 20, 1.82-1.70 (m, 2H), 1.01 (t 3H).

### (b) 2-Bromo-6-nitro-3-propoxypyridine

Prepared by a procedure analogous to that described above for 2-bromo-3-methoxy-6-nitropyridine using 2-bromo-3-propoxypyridine (2.20 g, 10.2 mmol) in place of 2-bromo-3-methoxypyridine. Yield: 1.58 g (59%) of slightly yellow solid.

¹H NMR (DMSO-d₆, 400 MHz) δ 8.38 (d, 1H), 7.79 (d, 1H), 4.23 (t, 2H), 1.87-1.75 (m, 2H), 1.02 (t, 3H).

### (c) 2-Amino-5-propoxypyridine

Prepared by a procedure analogous to that described above for 2-amino-5-methoxypyridine using 2-bromo-6-nitro-3-propoxypyridine (1.55 g, 5.94 mmol) in place of 2-bromo-3-methoxy-6-nitropyridine. Yield: 913 mg (100%) of white solid.

¹H NMR (DMSO-d₆, 400 MHz) δ 7.62 (d, 1H), 7.09 (dd, 1H), 6.40 (d, 1H), 5.41 (br. s, 2H), 3.81 (t, 2H), 1.71-1.59 (m, 2H), 0.94 (t, 3H).

### 2-Amino-5-butoxypyridine

### (a) 2-Bromo-3-butoxypyridine

Prepared by a procedure analogous to that described above for 2-bromo-3-ethoxypyridine using 1-iodobutane in place of iodoethane. Yield: 2.185 g (95%) of yellow oil.

¹H NMR (DMSO-d₆, 400 MHz) δ 7.94 (dd, 1H), 7.51 (dd, 1H), 7.39 (dd, 1H), 4.06 (t, 2H), 1.77-1.68 (m, 2H), 1.53-1.40 (m, 2H), 0.94 (t, 3H)

### (b) 2-Bromo-3-butoxy-6-nitro-3-propoxypyridine

Prepared by a procedure analogous to that described above for 2-bromo-3-methoxy-6-nitropyridine using 2-bromo-3-butoxypyridine (2.10 g, 9.13 mmol) in place of 2-bromo-3-methoxypyridine. Yield: 1.04 g (41%) of slightly yellow solid.

¹H NMR (DMSO-d₆, 400 MHz): δ 8.39 (d, 1H), 7.80 (d, 1H), 4.28 (t, 2H), 1.82-1.67 (m, 2H), 1.54-1.42 (m, 2H), 0.96 (t, 3H).

### (c) 2-Amino-5-butoxypyridine

Prepared by a procedure analogous to that described above for 2-amino-5-methoxypyridine using 2-bromo-3-butoxy-6-nitropyridine (1.03 g, 3.74 mmol) in place of 2-bromo-3-methoxy-6-nitropyridine. Yield: 501 mg (81%) of white solid.

¹H NMR (DMSO-d₆, 400 MHz) δ 7.63 (d, 1H), 7.09 (dd, 1H), 6.41 (d, 1H), 5.42 (br. s, 2H), 3.81 (t, 2H), 1.68-1.58 (m, 2H), 1.47-1.34 (m, 2H), 0.92 (t, 3H).

### 2-Amino-5-ethylpyridine

Diethylzinc (24 mL, of 1M solution in hexane, 24 mmol) was added dropwise to a solution of 2-amino-5-bromopyridine (2.0 g, 11.6 mmol) and Pd(dppf)Cl₂.CH₂Cl₂ (225 mg, 0.28 mmol) in degassed dioxane (45 mL). The mixture was stirred at rt for 2 h, then heated at reflux for 31 h and stirred at rt for 70 h under an argon atmosphere. The mixture was poured into NaCl (aq., sat; 150 mL) and extracted with EtOAc (4×100 mL). The combined extracts were washed with NaCl (aq., sat.; 100 mL), dried (Na₂SO₄) and concentrated. The crude product was purified by chromatography (EtOAc/heptane, then MeOH/EtOAc) to give the title compound (1.40 g, 99%).

¹H NMR (DMSO-d₆, 400 MHz) δ 7.74 (s, 1H), 7.25 (dd, 1H), 6.40 (d, 1H), 5.67 (br. s, 2H), 2.39 (q, 2H), 1.10 (t, 3H).

### 2-Amino-5-propylpyridine

Propylmagnesiumbromide (6 mL of a 2M solution in diethyl ether, 12 mmol) was added to a solution of zinc chloride (1M in diethyl ether, 6 mL, 6 mmol) under an argon atmosphere at 0° C. The solution was diluted with 1,4-dioxane (10 mL) and transferred into a suspension of 2-amino-5-bromopyridine (516 mg, 3 mmol) and Pd(dppf)Cl₂-CH₂Cl₂ (55 mg, 0.07 mmol) in 1,4-dioxane (5 mL). The mire was heated atreflux for 20 h. After cooling to rt the mixture was poured into water (50 mL) and NaHCO₃ (aq, 1M; 20 mL) was added. The mixture was extracted with EtOAc (3×50 mL) and the combined extracts washed with NaCl (aq., sat.; 50 mL), dried (Na₂SO₄) and concentrated in vacuo to give 575 mg of a dark oil, which was used without further purification.

¹H-NMR (CD₃OD, 400 MHz) δ 7.74 (d, 1H), 7.43 (d, 1H), 6.62 (d, 1H), 2.43 (t, 2H), 1.55-1.62 (m, 2H), 0.91 (t, 3H).

### 2-Amino-5-butylpyridine

Prepared by a procedure analogous to that described above for 2-amino-5-propylpyridine using butylmagnesiumchloride (2M in THF, 12 mL; 24 mmol) in place of propylmagnesiumbromide. The crude product was purified by chromatography (EtOAc/heptane) to give 405 mg (45%) of the title compound as brown solid.

¹H NMR (DMSO-d₆, 400 MHz) δ 7.71 (d, 1H), 7.21 (dd, 1H), 6.37 (d, 1H), 5.61 (br. s, 2H), 2.37 (t, 1H), 1.46 (p, 2H), 1.25-1.30 (m, 2H), 0.88 (t, 3H).

### 2-Amino-5-ethyl-6-methylpyridine

Prepared by a procedure analogous to that described above for 2-amino-5-ethylpyridine using 2-amino-5-bromo-6-methylpyridine (2.0 g, 10.7 mmol) in place of 2-amino-5-bromopyridine. The crude product was purified by chromatography (EtOAc/heptane) to give the title compound as brown crystals.

Yield: 0.74 g (51%).

¹H NMR (DMSO-d₆, 400 MHz) δ 7.06 (d, 1H), 6.21 (d, 1H), 5.51 (s, 2H), 2.40 (q, 2H), 2.21 (s, 3H), 1.06 (t, 3H).

### 2-Amino-5,6-dimethylpyridine

A solid mixture of 2-amino-5-bromo-6-methylpyridine (561 mg, 3.0 mmol), K₂CO₃ (1.24 g, 9.0 mmol) and Pd(dppf)Cl₂—CH₂Cl₂ (245 mg, 0.30 mmol) was added to a solution of trimethylboroxine (377 mg, 3.0 mmol) and water (1 mL) in 1,4-dioxane (10 mL). The mixture was heated at reflux for 3 h. After cooling to rt, the mixture was poured into water (50 mL) and the mixture extracted with diethyl ether (3×50 ml), the combined organic phases were dried (Na₂SO₄) and concentrated. The material was purified by chromatography (EtOAc/heptane) to give the title compound as black-brown solid. Yield: 244 mg (67%).

¹H NMR (DMSO-d₆, 400 MHz) δ 7.09 (d, 1H), 6.18 (d, 1H), 5.50 (br. s, 2H), 2.18 (s, 3H), 2.03 (s, 3H).

### EXAMPLES 1 TO 69

### General Procedures

### Method A

TBTU (1.1 mmol) was added to a solution of 1,2,3-triazole-4-carboxylic acid (113 mg, 1.0 mmol) and diisopropylethylamine (258 mg, 2 mmol) in anhydrous DMF (1 mL) and the mixture was stirred at rt for 10 min. The relevant arylamine (1.3 mmol) was added and the mixture was stirred at the indicated temperature for the indicated period of time. The resulting mixture was concentrated and water (20 mL) was added to the residue. The mixture was extracted with EtOAc (3×20 mL) and the combined extracts were washed with water (20 mL), dried (Na₂SO₄) and concentrated. The residue was purified by chromatography (eluent EtOAc/heptane) to give the title product.

### Method B

A mixture of 1,2,3-triazole-4-carboxylic acid (65 mg, 0.50 mmol), SOCl₂ (1 mL) and DMF (1 drop) was heated at 40° C. for 2 h. The mixture was concentrated and the residue was dried in vacuo. A mixture of the resulting solid, DMAP (83 mg, 0.68 mmol) and the relevant arylamine (2.0 mmol) in CH₂Cl₂ (5 mL) was sired at the indicated temperature for the indicated period of time and then concentrated. The residue was dissolved in EtOAc (20 mL), washed with HCl (aq, 2M, 2×5 mL) and NaCl (aq, sat 5 mL), dried (MgSO₄) and concentrated. The residue was purified by chromatography (eluent EtOAc/heptane, 1:1) to give the title product.

### Method C

Oxalyl chloride (0.58 mL, 6.6 mmol) was added dropwise to a mixture of 1,2,3-triazole-1-carboxylic acid (678 mg, 6.0 mmol), DMF (1.0 mL) and THF (30 mL) under an argon atmosphere at 0° C. The mixture was stirred at 0° C. for 2 h and transferred dropwise to a solution of the relevant arylamine (2.2 mmol) and DIPEA (0.76 mL, 4.4 mmol) in THF (1.0 mL) cooled to 0° C. The mixture was stirred at 0° C. for 30 min and heated to the indicated temperature for the indicated period of time. After cooling to rt the mixture was poured into a stirred rupture of EtOAc (30 mL) and water (30 mL). The organic phase was separated and concentrated. The residue was purified by chromatography (eluent EtOAc/heptane, 20-60%) and then crystallised from diethyl ether/heptane to give the title product

TABLE 1
Examples (Ex.) 1 to 69

| | | | Reaction conditions | | | |
|---|---|---|---|---|---|---|
| | | Prepared from | | | Temp | Yield |
| Ex | Name | arylamine | Method | Time h | ° C. | (%) |
| 1 | 1,2,3-Triazole-4- | 3-Aminoquinoline | A | 18 | 20 | 9 |
| | carboxylic acid | | | | | |
| | quinolin-3-ylamide | | | | | |
| 2 | 1,2,3-Triazole-4- | 2-Chloro-4-aniline | A | 18 | 20 | 4 |
| | carboxylic acid | | | | | |
| | (2-chloro-4-fluoro- | | | | | |
| | phenyl)amide | | | | | |
| 3 | 1,2,3-Triazole-4- | 2,4-Dichloro- | A | 18 | 20 | 5 |
| | carboxylic acid (2,4- | aniline | | | | |
| | dichlorophenyl)amide | | | | | |
| 4 | 1,2,3-Triazole-4- | 4-Fluoroaniline | A | 18 | 20 | 6 |
| | carboxylic acid | | | | | |
| | (4-fluorophenyl)amide | | | | | |
| 5 | 1,2,3-Triazole-4- | 4-Aminoquinoline | A | 144 | 22 | 10 |
| | carboxylic acid | | | | | |
| | quinolin-4-ylamide | | | | | |
| 6 | 1,2,3-Triazole-4- | 2,3,4-Trichloro- | A | 120 | 22 | 14 |
| | carboxylic acid (2,3,4- | aniline | | | | |
| | trichlorophenyl)amide | | | | | |
| 7 | 1,2,3-Triazole-4- | 2-Amino-5- | A | 18 | 20 | 1 |
| | carboxylic acid | chloropyridine | | | | |
| | (5-chloropyridin-2- | | | | | |
| | yl)amide | | | | | |
| 8 | 1,2,3-Triazole-4- | Aniline | A | 18 | 20 | 23 |
| | carboxylic acid | | | | | |
| | phenylamide | | | | | |
| 9 | 1,2,3-Triazole-4- | 3,4-Dichloro- | A | 18 | 20 | 33 |
| | carboxylic acid (3,4- | aniline | | | | |
| | dichlorophenyl)amide | | | | | |
| 10 | 1,2,3-Triazole-4- | 2-Chloroaniline | A | 18 | 20 | 8 |
| | carboxylic acid | | | | | |
| | (2-chlorophenyl)amide | | | | | |
| 11 | 1,2,3-Triazole-4- | 2-Amino-5- | B | 72 | 20 | 44 |
| | carboxylic acid | trifluoromethyl- | | | | |
| | (5-trifluoromethylpyridin- | pyridine | | | | |
| | 2-yl)amide | | | | | |
| 12 | 1,2,3-Triazole-4- | 2,4,5-Trichloro- | A | 18 | 50 | 8 |
| | carboxylic acid (2,4,5- | aniline | | | | |
| | trichlorophenyl)amide | | | | | |
| 13 | 1,2,3-Triazole-4- | 2,4-Xylidine | A | 18 | 22 | 76 |
| | carboxylic acid (2,4- | | | | | |
| | dimethylphenyl)amide | | | | | |
| 14 | 1,2,3-Triazole-4- | 2,5-Dichloro- | A | 18 | 22 | 19 |
| | carboxylic acid (2,5- | aniline | | | | |
| | dichlorophenyl)amide | | | | | |
| 15 | 1,2,3-Triazole-4- | 2-Amino-5- | B | 18 | 20 | 23 |
| | carboxylic acid | fluoropyridine | | | | |
| | (5-fluoropyridin-2-yl)- | | | | | |
| | amide | | | | | |
| 16 | 1,2,3-Triazole-4- | 2,4-Dimethoxy- | A | 48 | 22 | 19 |
| | carboxylic acid (2,4- | aniline | | | | |
| | dimethoxyphenyl)amide | | | | | |
| 17 | 1,2,3-Triazole-4- | 4-Chloro-2,5- | A | 24 | 60¹ | 35 |
| | carboxylic acid | dimethoxyaniline | | | | |
| | (4-chloro-2,5- | | | | | |
| | dimethoxyphenyl)amide | | | | | |
| 18 | 1,2,3-Triazole-4- | 4-Chloro-m- | A | 24 | 60¹ | 47 |
| | carboxylic acid | toluidine | | | | |
| | (4-chloro-3-methyl- | | | | | |
| | phenyl)amide | | | | | |
| 19 | 1,2,3-Triazole-4- | Cumidine | A | 24 | 60¹ | 53 |
| | carboxylic acid (4-iso- | | | | | |
| | propylphenyl)amide | | | | | |
| 20 | 1,2,3-Triazole-4- | 4-Amino-N,N- | A | 18 | 80 | 25 |
| | carboxylic acid | diethylbenzene- | | | | |
| | (4-diethylsulfamoyl- | sulfonamide | | | | |
| | pheny) amide | | | | | |
| 21 | 1,2,3-Triazole-4- | 2-Aminoquino- | A | 18 | 80 | 4 |
| | carboxylic acid | xaline | | | | |
| | quinoxalin-2-ylamide | | | | | |
| 22 | 1,2,3-Triazole-4- | 4-Aminobenzene- | A | 24 | 60¹ | 7 |
| | carboxylic acid (4- | sulfonamide | | | | |
| | sulfamoylphenyl)amide | | | | | |
| 23 | 1,2,3-Triazole-4- | 4-Chloro-o- | A | 24 | 85 | 33 |
| | carboxylic acid | anisidine | | | | |
| | (4-chloro-2-methoxy- | | | | | |
| | phenyl)amide | | | | | |
| 24 | 1,2,3-Triazole-4- | 2,4-Dichloro-m- | A | 24 | 85 | 27 |
| | carboxylic acid | toluidine | | | | |
| | (2,4-dichloro-3-methyl- | | | | | |
| | phenyl)amide | | | | | |
| 25 | 1,2,3-Triazole-4- | 4-Amino-N- | A | 24 | 85 | 31 |
| | carboxylic acid | methylbenzene- | | | | |
| | (4-methylsulfamoyl- | sulfonamide | | | | |
| | phenyl)amide | | | | | |
| 26 | 1,2,3-Triazole-4- | 4-Amino-N,N- | A | 24 | 85 | 23 |
| | carboxylic acid | dimethylbenzene- | | | | |
| | (4-dimethylsulfamoyl- | sulfonamide | | | | |
| | phenyl)amide | | | | | |
| 27 | 1,2,3-Triazole-4- | 2,4-Dichloro-6- | A | 48 | 85 | 24 |
| | carboxylic acid | methylaniline | | | | |
| | (2,4-dichloro-6-methyl- | | | | | |
| | phenyl)amide | | | | | |
| 28 | 1,2,3-Triazole-4- | 3-Amino-6-fluoro- | A | 18 | 85 | 26 |
| | carboxylic acid | quinoline | | | | |
| | (6-fluoroquinolin-3-yl)- | | | | | |
| | amide | | | | | |
| 29 | 1,2,3-Triazole-4- | 3-Amino-8-fluoro- | A | 18 | 85 | 46 |
| | carboxylic acid | quinoline | | | | |
| | (8-fluoroquinolin-3-yl)- | | | | | |
| | amide | | | | | |
| 30 | 1,2,3-Triazole-4- | 3-Amino-8-chloro- | A | 18 | 85 | 8 |
| | carboxylic acid | quinoline | | | | |
| | (8-chloroquinolin-3-yl)- | | | | | |
| | amide | | | | | |
| 31 | 1,2,3-Triazole-4- | 3-Amino-7-fluoro- | A | 18 | 85 | 60 |
| | carboxylic acid | quinoline | | | | |
| | (7-fluoroquinolin-3-yl)- | | | | | |
| | amide | | | | | |
| 32 | 1,2,3-Triazole-4- | 2-Chloro-4,6- | C | 16 | 60 | 28 |
| | carboxylic acid | difluoroaniline | | | | |
| | (2-chloro-4,6-difluoro- | | | | | |
| | phenyl)amide | | | | | |
| 33 | 1,2,3-Triazole-4- | 2,3-Dichloro- | A | 2 | 80 | 29 |
| | carboxylic acid (2,3- | aniline | | | | |
| | dichlorophenyl)amide | | | | | |
| 34 | 1,2,3-Triazole-4- | 2-Amino-5- | B | 18 | 80 | 5 |
| | carboxylic acid | chlorothiazole | | | | |
| | (5-chlorothiazol-2-yl)- | | | | | |
| | amide | | | | | |
| 35 | 1,2,3-Triazole-4- | 2-Amino-5- | A | 16 | 60 | 35 |
| | carboxylic acid | bromopyridine | | | | |
| | (5-bromopyridin-2-yl)- | | | | | |
| | amide | | | | | |
| 36 | 1,2,3-Triazole-4- | 2,4-Bis(trifluoro- | C | 2 | 60 | 3 |
| | carboxylic acid | methyl)aniline | | | | |
| | [2,4-bis(trifluoromethyl)phenyl]amide | | | | | |
| 37 | 1,2,3-Triazole-4- | 2-Amino-5- | A | 96 | 100 | 16 |
| | carboxylic acid (5- | nitropyridine | | | | |
| | nitropyridin-2-yl)amide | | | | | |
| 38 | 1,2,3-Triazole-4- | 2-Amino-N- | A | 72 | 100 | 15 |
| | carboxylic acid | methylbenzene- | | | | |
| | [2-(methylsulfamoyl)- | sulfonamide | | | | |
| | phenyl]amide | | | | | |
| 39 | 1,2,3-Triazole-4- | 2,4,6-Trifluoro- | C | ⅓ | 20 | 21 |
| | carboxylic acid (2,4,6- | aniline | | (20 min) | | |
| | trifluorophenyl)amide | | | | | |
| 40 | 1,2,3-Triazole-4- | 2-Amino-6- | A | 20 | 100 | 33 |
| | carboxylic acid | methoxypyridine | | | | |
| | (6-methoxypyridin-2- | | | | | |
| | yl)amide | | | | | |
| 41 | 1,2,3-Triazole-4- | 2-Amino-6-bromo- | A | 16 | 80 | 7 |
| | carboxylic acid | pyridine | | | | |
| | (6-bromopyridin-2-yl)- | | | | | |
| | amide | | | | | |
| 42 | 1,2,3-Triazole-4- | 2,6-Dichloro-4- | B | 16 | 60 | 25 |
| | carboxylic acid | fluoroaniline | | | | |
| | (2,6-dichloro- | | | | | |
| | 4-fluorophenyl)amide | | | | | |
| 43 | 1,2,3-Triazole-4- | 2-Amino-4- | A | 3 | 80 | 6 |
| | carboxylic acid | trifluoromethyl- | | | | |
| | (4-trifluoromethyl- | pyridine | | | | |
| | pyridin-2-yl)amide | | | | | |
| 44 | 1,2,3-Triazole-4- | 2-Amino-4- | A | 67 | 80 | 33 |
| | carboxylic acid | methylpyridine | | | | |
| | (4-methylpyridin-2-yl)- | | | | | |
| | amide | | | | | |
| 45 | 1,2,3-Triazole-4- | 3-Amino-2,5- | A | 48 | 80 | 9 |
| | carboxylic acid | dichloro-pyridine | | | | |
| | (2,5-dichloropyridin-3- | | | | | |
| | yl)amide | | | | | |
| 46 | 1,2,3-Triazole-4- | 2-Amino-5- | A | 67 | 80 | 25 |
| | carboxylic acid | methylpyridine | | | | |
| | (5-methylpyridin-2-yl)- | | | | | |
| | amide | | | | | |
| 47 | 1,2,3-Triazole-4- | 2-Amino-5-ethyl- | A | 3 | 80 | 58 |
| | carboxylic acid (5-ethyl- | 6-methylpyridine | | | | |
| | 6-methylpyridin-2-yl)- | | | | | |
| | amide | | | | | |
| 48 | 1,2,3-Triazole-4-carboxylic | 2-Amino-3-chloro- | A | 67 | 80 | 3 |
| | acid (3-chloro-5- | 5-trifluoromethyl- | | | | |
| | trifluoromethylpyridin- | pyridine | | | | |
| | 2-yl)amide | | | | | |
| 49 | 1,2,3-Triazole-4- | 2,6-Dichloro-4- | C | 15 | 60 | 15 |
| | carboxylic acid (2,6- | trifluoromethyl- | | | | |
| | dichloro-4-trifluoro- | aniline | | | | |
| | methylphenyl)amide | | | | | |
| 50 | 1,2,3-Triazole-4- | 2-Amino-5,6- | A² | 70 | 20 | 37 |
| | carboxylic acid | dimethylpyridine | | | | |
| | (5,6-dimethylpyridin-2- | | | | | |
| | yl)amide | | | | | |
| 51 | 1,2,3-Triazole-4-carboxylic | 3-Amino-5- | A | 48 | 80 | 43 |
| | acid (5-methyl- | methylpyridine | | | | |
| | pyridin-3-yl)amide | | | | | |
| 52 | 1,2,3-Triazole-4-carboxylic | 2-Amino-5- | A | 44 | 80 | 59 |
| | acid (5-methoxy- | methoxypyridine | | | | |
| | pyridin-2-yl)amide | | | | | |
| 53 | 1,2,3-Triazole-4-carboxylic | 2-Amino-5,6- | A | 66 | 80 | 43 |
| | acid (5,6-dimethoxypyridine- | dimethoxy- | | | | |
| | 2-yl)amide | pyridine | | | | |
| 54 | 1,2,3-Triazole-4-carboxylic | 2-Amino-6- | A | 67 | 80 | 48 |
| | acid (6-methyl- | methylpyridine | | | | |
| | pyridin-2-yl)amide | | | | | |
| 55 | 1,2,3-Triazole-4- | 2-Amino-4,6- | A | 67 | 80 | 45 |
| | carboxylic acid | dimethylpyridine | | | | |
| | (4,6-dimethylpyridin-2- | | | | | |
| | yl)amide | | | | | |
| 56 | 1,2,3-Triazole-4-carboxylic | 2-Amino-3,5- | A | 67 | 80 | 4 |
| | acid (3,5-dichloro- | dichloropyridine | | | | |
| | pyridin-2-yl)amide | | | | | |
| 57 | 1,2,3-Triazole-4- | 2-Amino-4- | C | 1 | 68 | 6 |
| | carboxylic acid | methylpyrimidine | | | | |
| | (4-methylpyrimidin-2- | | | | | |
| | yl)amide | | | | | |
| 58 | 1,2,3-Triazole-4- | 2-Amino- | C | 18 | 25 | 6 |
| | carboxylic acid | pyrimidine | | | | |
| | (pyrimidin-2-yl)amide | | | | | |
| 59 | 1,2,3-Triazole-4-carboxylic | 2-Amino-3- | A | 48 | 100 | 41 |
| | acid (3-methoxy- | methoxypyridine | | | | |
| | pyridin-2-yl)amide | | | | | |
| 60 | 1,2,3-Triazole-4-carboxylic | 2-Amino-5- | A | 44 | 80 | 36 |
| | acid (5-butoxy- | butoxypyridine | | | | |
| | pyridin-2-yl)amide | | | | | |
| 61 | {6-[(1,2,3-Triazole-4- | (6-Amino-pyridin- | A | 44 | 80 | 26 |
| | carbonyl)amino]pyridin- | 3-yl)-carbamic | | | | |
| | 3-yl}carbamic acid tert- | acid tert-butyl | | | | |
| | butyl ester | ester | | | | |
| 62 | 1,2,3-Triazole-4- | 2-Amino-N,N- | A | 66 | 80 | 12 |
| | carboxylic acid | dimethylbenzenesulfonamide | | | | |
| | [2-(N,N-dimethylsulfamoyl)phenyl]- | | | | | |
| | amide | | | | | |
| 63 | 1,2,3-Triazole-4-carboxylic | 2-Amino-5- | A | 66 | 80 | 42 |
| | acid (5-ethoxy- | ethoxypyridine | | | | |
| | pyridin-2-yl)amide | | | | | |
| 64 | 1,2,3-Triazole-4-carboxylic | 2-Amino-5- | A | 66 | 80 | 40 |
| | acid (5-propoxy- | propoxypyridine | | | | |
| | pyridin-2-yl)amide | | | | | |
| 65 | 1,2,3-Triazole-4-carboxylic | 3-Amino-6- | A | 48 | 80 | 42 |
| | acid (6-methoxy-5- | methoxy-5- | | | | |
| | methylpyridin-3-yl)- | methylpyridine | | | | |
| | amide | | | | | |
| 66 | 1,2,3-Triazole-4-carboxylic | 3-Amino-5- | A | 48 | 80 | 22 |
| | acid (5-phenyl- | phenylpyridine | | | | |
| | pyridin-3-yl)amide | | | | | |
| 67 | 1,2,3-Triazole-4-carboxylic | 2-Amino-5- | A² | 18 | 25 | 16 |
| | acid (5-propyl- | propylpyridine | | | | |
| | pyridin-2-yl)amide | | | | | |
| 68 | 1,2,3-Triazole-4- | 2-Amino-5- | A | 18 | 80 | 59 |
| | carboxylic acid (5- | ethylpyridine | | | | |
| | ethylpyridin-2-yl)amide | | | | | |
| 69 | 1,2,3-Triazole-4- | 2-Amino-6- | B | 18 | 60 | 7 |
| | carboxylic acid (6- | trifluoromethyl- | | | | |
| | trifluoromethylpyridin- | pyridine | | | | |
| | 2-yl)amide | | | | | |

¹The reaction mixture was stirred at rt for 3 days before the heating at the indicated temperature for the indicated time
²Bromotripyrrolidinophosphonium hexafluorophosphate (PyBroP, 470 mg, 1.0 mmol) was used instead of TBTU

TABLE 2
Physical properties of the compounds of Examples 1-69

| | | MS | |
|---|---|---|---|
| | | (M⁺ + 1), | |
| Ex. | M.W. | m/z | ¹H NMR (DMSO-d₆, 400 MHz), δ |
| 1 | 239.24 | 240 | 14.9 (br. s, 1H), 10.92 (s, 1H), 9.22 (d, 1H), 8.84 (d, |
| | | | 1H), 8.61 (br. s, 1H), 7.97 (t, 2H), 7.68 (t, 1H), |
| | | | 7.59 (t, 1H) |
| 2 | 240.63 | 241 | 9.17 (s, 1H), 8.42 (dd, 1H), 8.26 (s, 1H), 7.13 (dd, |
| | | | 1H), 7.00 (ddd, 1H)¹ |
| 3 | 257.08 | 257 | 9.29 (s, 1H), 8.55 (s, 1H), 7.89 (d, 1H), 7.69 (d, 1H), |
| | | | 7.43 (dd, 1H) |
| 4 | 206.18 | 207 | 10.45 (s, 1H), 8.80 (s, 1H), 7.81-7.77 (m, 2H), |
| | | | 7.17-7.11 (m, 2H) |
| 5 | 239.24 | 240 | 10.73 (s, 1H), 8.89 (d, 1H), 8.69 (s, 1H), 8.18 (d, 1H), |
| | | | 8.05 (d, 1H), 8.00 (d, 1H), 7.81 (t, 1H), 7.67 (t, 1H) |
| 6 | 291.52 | 291 | 10.13 (s, 1H), 8.64 (s, 1H), 7.92 (s, 1H), 7.71 (s, 1H) |
| 7 | 223.62 | 224 | 10.41 (br. s, 1H), 8.67 (s, 1H), 8.42 (d, 1H), 8.18 (d, |
| | | | 1H), 7.96 (dd, 1H) |
| 8 | 188.19 | 189 | 10.36 (s, 1H), 8.50 (s, 1H), 7.92 (s, 1H), 7.79 (d, 2H), |
| | | | 7.31 (t, 2H), 7.07 (t, 1H) |
| 9 | 257.08 | 257 | 10.73 (s, 1H), 8.55 (s, 1H), 8.18 (d, 1H), 7.92 (s, 1H), |
| | | | 7.82 (dd, 1H), 7.58 (d, 1H) |
| 10 | 222.63 | 223 | 9.88 (s, 1H), 8.59 (s, 1H), 7.94 (d, 1H), 7.54 (dd, 1H), |
| | | | 7.37 (dt, 1H), 7.22 (dt, 1H) |
| 11 | 257.18 | 258 | 8.67 (m, 1H), 8.44 (m, 2H), 8.12 (dd, 1H)² |
| 12 | 291.52 | 291 | 10.00 (s, 1H), 8.63 (br. s, 1H), 8.23 (s, 1H), 7.99 (s, |
| | | | 1H) |
| 13 | 216.24 | 217 | 9.77 (s, 1H), 8.46 (s, 1H), 7.29 (d, 1H), 7.04 (s, 1H), |
| | | | 6.98 (d, 1H), 2.25 (s, 3H), 2.18 (s, 3H) |
| 14 | 257.08 | 257 | 9.93 (s, 1H), 8.64 (s, 1H), 8.08 (d, 1H), 7.59 (d, 1H), |
| | | | 7.31 (dd, 1H) |
| 15 | 207.17 | 208 | 15.73 (br. s, 1H), 10.35 (br. s, 1H), 8.69 (br. s, 1H), |
| | | | 8.40 (d, 1H), 8.13-8.21 (m, 1H), 7.82 (ddd, 1H) |
| 16 | 248.24 | 249 | 9.29 (s, 1H), 8.53 (s, 1H), 7.98 (d, 1H), 6.67 (d, 1H), |
| | | | 6.53 (dd, 1H), 3.87 (s, 3H), 3.75 (s, 3H) |
| 17 | 282.69 | 283 | 9.46 (1H, s), 8.60 (1H, br. s), 8.14 (1H, s), 7.23 (1H, |
| | | | s), 3.89 (3H, s), 3.81 (3H, s) |
| 18 | 236.66 | 237 | 10.44 (1H, s), 8.51 (1H, s), 7.81 (1H, d), 7.66 (1H, |
| | | | dd), 7.35 (1H, d), 2.32 (3H, s) |
| 19 | 230.27 | 231 | 10.28 (1H, s), 8.48 (1H, s), 7.69 (2H, d), 7.19 (2H, d), |
| | | | 2.85 (1H, septet), 1.20 (3H, s), 1.18 (3H, s) |
| 20 | 323.38 | 324 | 10.79 (s, 1H), 8.57 (s, 1H), 8.03 (d, 2H), 7.74 (d, 2H), |
| | | | 3.13 (q, 4H), 1.03 (t, 6H) |
| 21 | 240.23 | 241 | 15.85 (br.s, 1H), 11.10 (br.s, 1H), 9.67 (s, 1H), |
| | | | 8.77 (s, 1H), 8.10 (dd, 1H), 8.96 (dd, 1H), 7.85 (dt, 1H), |
| | | | 7.77 (dt, 1H) |
| 22 | 267.27 | 268 | 15.9-15.6 (1H, br. s), 10.71 (1H, s), 8.56 (1H, s), |
| | | | 8.00 (2H, d), 7.79 (2H, d), 7.27 (2H, s) |
| 23 | 252.66 | 253 | 15.9-15.6 (1H, br. s), 9.48 (1H, s), 8.63 (1H, s), |
| | | | 8.21 (1H, d), 7.21 (1H, d), 7.06 (1H, dd), 3.95 (3H, s) |
| 24 | 271.11 | 271 | 15.9-15.7 (1H, br. s), 9.94 (1H, s), 8.63 (1H, s), |
| | | | 7.88 (1H, d), 7.50 (1H, d), 2.48 (3H, s) |
| 25 | 281.30 | 282 | 15.8-15.7 (1H, br. s), 10.78 (1H, s), 8.58 (1H, s), |
| | | | 8.04 (2H, d), 7.76 (2H, d), 7.33 (1H, q), 2.41 (3H, d) |
| 26 | 295.32 | 296 | 15.9-15.7 (1H, br. s), 10.85 (1H, s), 8.61 (1H, s), |
| | | | 8.12 (2H, d), 7.73 (2H, d), 2.61 (6H, s) |
| 27 | 271.11 | 271 | 15.8-15.6 (1H, br. s), 10.16 (1H, s), 8.51 (1H, s), |
| | | | 7.56 (1H, d), 7.43 (1H, d), 2.23 (3H, s) |
| 28 | 257.23 | 258 | 15.91-15.67 (1H, br. s), 10.97 (1H, s), 9.20 (1H, d), |
| | | | 8.87 (1H, d), 8.73-8.54 (1H, br. s), 8.02 (1H, dd), |
| | | | 7.79 (1H, dd), 7.56 (1H, ddd) |
| 29 | 257.23 | 258 | 15.97-15.65 (1H, br. s), 11.04 (1H, s), 9.28 (1H, d), |
| | | | 8.94 (1H, dd), 8.74-8.54 (1H, br. s), 7.80 (1H, br. d), |
| | | | 7.58 (1H, ddd), 7.49 (1H, dd) |
| 30 | 273.68 | 274 | 15.95-15.60 (1H, br. s), 11.06 (1H, s), 9.33 (1H, d), |
| | | | 8.96 (1H, d), 8.69-8.54 (1H, br. s), 7.96 (1H, dd), |
| | | | 7.84 (1H, dd), 7.57 (1H, t) |
| 31 | 257.23 | 258 | 16.26-15.24 (1H, br. s), 10.95 (1H, s), 9.25 (1H, d), |
| | | | 8.90 (1H, d), 8.65-8.57 (1H, br. s), 8.08 (1H, dd), |
| | | | 7.72 (1H, dd), 7.75 (1H, dt) |
| 32 | 258.61 | 259 | 15.73 (br. s, 1H), 10.26 (s, 1H), 8.55 (br. s, 1H), |
| | | | 7.54-7.44 (m, 2H) |
| 33 | 257.08 | 257 | 15.81 (br. s, 1H), 10.07 (s, 1H), 8.63 (s, 1H), 7.94 (dd, |
| | | | 1H), 7.51 (dd, 1H), 7.42 (dd, 1H) |
| 34 | 229.65 | 230 | 8.46 (br. s, 1H), 7.39 (s, 1H)² |
| 35 | 268.07 | 268 | 15.83 (br. s, 1H), 10.43 (s, 1H), 8.69 (s, 1H), 8.51 (d, |
| | | | 1H), 8.14 (d, 1H), 8.11 (dd, 1H) |
| 36 | 324.18 | 325 | 15.87 (br. s, 1H), 10.15 (s, 1H), 8.69 (s, 1H), |
| | | | 8.23-8.11 (m, 3H) |
| 37 | 234.17 | 235 | 16.40-15.17 (br. s, 1H), 11.07-10.93 (br. s, 1H), |
| | | | 9.22 (d, 1H), 8.82-8.73 (br. s, 1H), 8.68 (dd, 1H), 8.40 (dd, |
| | | | 1H) |
| 38 | 281.29 | 282 | 16.29-15.29 (br. s, 1H), 10.84 (s, 1H), 8.62 (s, 1H), |
| | | | 8.55 (dd, 1H), 7.87-7.79 (br. s, 1H), 7.82 (dd, 1H), |
| | | | 7.70 (ddd, 1H), 7.34 (ddd, 1H), 2.46 (br. s, 3H) |
| 39 | 242.16 | 243 | 15.78 (br. s, 1H), 10.22 (s, 1H), 8.54 (s, 1H), |
| | | | 7.37-7.26 (m, 2H) |
| 40 | 219.20 | 220 | 15.88-15.70 (br. s, 1H), 9.88-9.75 (br. s, 1H), |
| | | | 8.73-8.62 (br. s, 1H), 7.77-7.75 (m, 2H), 6.60 (dd, 1H), |
| | | | 3.87 (s, 3H) |
| 41 | 268.07 | 268 | 15.74 (br. s, 1H), 10.61 (br. s, 1H), 8.70 (s, 1H), |
| | | | 8.17 (d, 1H), 7.81 (dd, 1H), 7.43 (d, 1H) |
| 42 | 275.07 | 275 | 15.72 (br. s, 1H), 10.37 (s, 1H), 8.54 (s, 1H), 7.66 (d, |
| | | | 2H) |
| 43 | 257.17 | 258 | 15.82 (br. s, 1H), 10.74 (s, 1H), 8.72 (s, 1H), 8.69 (d, |
| | | | 1H), 8.49 (s, 1H), 7.57 (d, 1H) |
| 44 | 203.20 | 204 | 15.85-15.61 (br. s, 1H), 10.10-9.91 (br. s, 1H), |
| | | | 8.65 (s, 1H), 8.22 (d, 1H), 8.06 (dd, 1H), 7.68 (dd, 1H), |
| | | | 2.28 (s, 3H) |
| 45 | 258.06 | 258 | 16.09-15.58 (br. s, 1H), 10.07 (s, 1H), 8.74-8.62 (br. |
| | | | s, 1H), 8.52 (d, 1H), 8.38 (d, 1H) |
| 46 | 203.20 | 204 | 15.81-15.67 (br. s, 1H), 10.00 (br. s, 1H), 8.67 (s, 1H), |
| | | | 8.23 (d, 1H), 8.02 (dd, 1H), 7.03 (dd, 1H), 2.36 (s, |
| | | | 3H) |
| 47 | 231.25 | 232 | 15.77 (br. s, 1H), 9.94 (br. s, 1H), 8.66 (s, 1H), |
| | | | 7.94 (d, 1H), 7.61 (d, 1H), 2.60 (q, 2H), 2.43 (s, 3H), |
| | | | 1.17 (t, 3H) |
| 48 | 291.92 | 292 | 15.93-15.66 (br. s, 1H), 10.99-10.83 (br. s, 1H), |
| | | | 8.89 (m, 1H), 8.70-8.57 (br. s, 1H), 8.61 (d, 1H) |
| 49 | 325.07 | 325 | 15.78 (br. s, 1H), 10.66 (br. s, 1H), 8.57 (br. s, 1H), |
| | | | 8.07 (s, 2H) |
| 50 | 217.23 | 218 | 15.83 (br. s, 1H), 9.92 (br. s, 1H), 8.63 (s, 1H), |
| | | | 7.79 (d, 1H), 7.58 (d, 1H), 7.58 (d, 1H), 2.39 (s, 3H), |
| | | | 2.23 (s, 3H) |
| 51 | 203.20 | 204 | 15.94-15.51 (br. s, 1H), 10.59 (s, 1H), 8.79 (d, 1H), |
| | | | 8.55 (br. s, 1H), 8.16 (m, 1H), 8.07 (m, 1H), 2.30 (s, |
| | | | 3H) |
| 52 | 219.20 | 220 | 16.20-15.27 (br. s, 1H), 10.09 (br. s, 1H), 8.63 (s, 1H), |
| | | | 8.11-8.07 (m, 2H), 7.50 (dd, 1H), 3.83 (s, 3H) |
| 53 | 249.23 | 250 | 15.98-15.46 (br. s, 1H), 9.81-9.58 (br. s, 1H), |
| | | | 8.71-8.53 (br. s, 1H), 7.65 (d, 1H), 7.38 (d, 1H), 3.89 (s, |
| | | | 3H), 3.78 (s, 3H) |
| 54 | 203.20 | 204 | 16.29-15.28 (br. s, 1H), 10.21-9.87 (br. s, 1H), |
| | | | 8.67 (s, 1H), 7.99 (d, 1H), 7.74 (dd, 1H), 7.04 (d, 1H), |
| | | | 2.44 (s, 3H) |
| 55 | 217.23 | 218 | 15.93-15.58 (br. s, 1H), 10.01-9.72 (br. s, 1H), |
| | | | 8.66 (s, 1H), 7.84 (s, 1H), 6.89 (s, 1H), 2.88 (s, 3H), |
| | | | 2.84 (s, 3H) |
| 56 | 258.06 | 258 | 15.91-15.56 (br. s, 1H), 10.79-10.69 (br. s, 1H), |
| | | | 8.62-8.51 (br. s, 1H), 8.55 (d, 1H), 8.36 (d, 1H) |
| 57 | 204.19 | 205 | 15.23 (br. s, 1H), 10.37 (br. s, 1H), 8.61 (br. s, 1H), |
| | | | 8.58 (d, 1H), 7.16 (d, 1H), 2.45 (s, 3H) |
| 58 | 190.16 | 191 | 15.7 (br. s, 1H), 10.47 (s, 1H), 8.74 (d, 2H), 8.65 (s, |
| | | | 1H), 7.28 (dd, 1H) |
| 59 | 219.20 | 220 | 15.83-15.51 (br. s, 1H), 10.07-10.00 (br. s, 1H), |
| | | | 8.66-8.43 (br. s, 1H), 8.01 (dd, 1H), 7.52 (dd, 1H), |
| | | | 7.28 (dd, 1H), 3.85 (s, 3H) |
| 60 | 261.28 | 262 | 16.23-15.42 (br. s, 1H), 10.27-9.90 (br. s, 1H), |
| | | | 8.63 (s, 1H), 8.10-8.06 (m, 2H), 7.50 (dd, 1H), 4.05 (dd, |
| | | | 2H), 1.76-1.66 (m, 2H), 1.51-1.38 (m, 2H), 0.95 (t, |
| | | | 3H) |
| 61 | 304.30 | 305 | 16.13-15.32 (br. s, 1H), 10.25-9.97 (br. s, 1H), |
| | | | 9.54 (s, 1H), 8.63 (s, 1H), 8.44 (d, 1H), 8.07 (d, 1H), |
| | | | 7.90 (dd, 1H), 1.48 (s, 9H) |
| 62 | 295.32 | 296 | 16.10-15.58 (br. s, 1H), 10.90 (s, 1H), 8.73-8.55 (br. |
| | | | s, 1H), 8.62 (dd, 1H), 7.83 (dd, 1H), 7.76 (ddd, 1H), |
| | | | 7.38 (ddd, 1H), 2.69 (s, 6H) |
| 63 | 233.23 | 234 | 15.97-15.52 (br. s, 1H), 10.27-9.90 (br. s, 1H), |
| | | | 8.63 (s, 1H), 8.10-8.05 (m, 2H), 7.50 (dd, 1H), 4.10 (q, |
| | | | 2H), 1.34 (t, 3H) |
| 64 | 247.25 | 248 | 15.93-15.34 (br. s, 1H), 10.19-9.93 (br. s, 1H), |
| | | | 8.63 (s, 1H), 8.10-8.05 (m, 2H), 7.50 (dd, 1H), 4.00 (t, 2H), |
| | | | 1.77-1.70 (m, 2H), 0.99 (t, 3H) |
| 65 | 233.23 | 234 | 15.91-15.36 (br. s, 1H), 10.42 (s, 1H), 8.51 (br. s, 1H), |
| | | | 8.38 (d, 1H), 7.94 (d, 1H), 3.87 (s, 3H), 2.16 (s, 3H) |
| 66 | 265.27 | 266 | 16.28-15.10 (br. s, 1H), 10.76 (s, 1H), 9.03 (d, 1H), |
| | | | 8.63 (d, 1H), 8.58 (br. s, 1H), 8.51 (dd, 1H), |
| | | | 7.74-7.68 (m, 2H), 7.54-7.40 (m, 3H) |
| 67 | 231.25 | 232 | 15.74 (br. s, 1H), 10.07 (br. s, 1H), 8.64 (s, 1H), |
| | | | 8.22 (d, 1H), 8.08 (d, 1H), 7.70 (dd, 1H), 2.54 (q, 2H), |
| | | | 1.58-1.63 (m, 2H), 0.90 (t, 3H) |
| 68 | 217.23 | 218 | 15.76 (br. s, 1H), 10.09 (br. s, 1H), 8.66 (s, 1H), |
| | | | 8.24 (d, 1H), 8.09 (d, 1H), 7.73 (dd, 1H), 2.61 (q, 2H), |
| | | | 1.20 (t, 3H) |
| 69 | 257.17 | 258 | 8.52 (d, 1H), 8.43 (br. s, 1H), 8.03 (dd, 1H), 7.54 (d, |
| | | | 1H)² |

¹Run in CDCl₃, 400 MHz
²Run in CD₃OD, 400 MHz

### EXAMPLES 70-78

### General Procedure

### (a) 3-[2-(Trimethylsilyl)ethoxymethyl]-1,2,3-triazole-4-carboxylic Acid Aryl-Amides

Butyllithium (1.6 M in hexanes, 1.1 mL, 1.7 mmol) was added dropwise to a solution of 1-[2-(trimethylsilyl)ethoxymethyl]-1,2,3-triazole (3:1 mixture of the isomers, prepared as described hereinbefore, 300 mg, 1.5 mmol) in TH (20 mL) cooled to −20° C. The mixture was stirred at −20° C. for 30 min and cooled to −78° C. A solution of the relevant arylisocyanate (2.0 mmol) in THF (5 mL) was added dropwise and the mixture was stirred at −78° C. for 2 h, allowed to warm to rt and then stirred at rt for 18 h. Et₂O (20 mL) and NH₄Cl (aq, sat, 10 mL) were added and the layers were separated. The aqueous phase was extracted with Et₂O (2×20 mL) and the combined extracts were dried (Na₂SO₄) and concentrated. The residue was purified by chromatography (eluent EtOAc/heptane) to give the sub-title products as white or yellow powders (Intermediates (a) 32 to 40).

### (b) 1,2,3-Triazole-4-carboxylic Acid Arylamides

A mixture of the relevant 3-(2-trimethylsilylethoxymethyl)-1,2,3-triazole-4-carboxylic acid arylamide (1.0 mmol) and HCl (2.7 M in EtOH, 1.51 mL) was stirred at rt for 20 min and concentrated. The residue was purified by chromatography (eluent EtOAc/heptane) to give the title products as white or yellow powders (Examples 32(b) to 40(b)).

TABLE 3
Intermediates (a) 32 to 40 and Examples (Ex.) (b) 70 to 78

| | | | Arylisocyanate | |
|---|---|---|---|---|
| Ex. | Step | Name | in Step (a) | Yield % |
| 70 | (a) | 3-[2-(Trimethylsilyl)ethoxymethyl]- | 2,4,6- | 41 |
| | | 1,2,3-triazole-4-carboxylic acid 2,4,6- | Trichlorophenyl- | |
| | | trichlorophenylamide | isocyanate | |
| | (b) | 1,2,3-Triazole-4-carboxylic acid | | 56 |
| | | (2,4,6-trichlorophenyl)amide | | |
| 71 | (a) | 3-[2-(Trimethylsilyl)ethoxymethyl]- | 2- | 59 |
| | | 1,2,3-triazole-4-carboxylic acid 2- | (Trifluoromethyl)- | |
| | | (trifluoromethyl)phenylamide | phenyliso- | |
| | | | cyanate | |
| | (b) | 1,2,3-Triazole-4-carboxylic acid 2- | | 39 |
| | | (trifluoromethyl)phenylamide | | |
| 72 | (a) | 3-[2-(Trimethylsilyl)ethoxymethyl]- | 4-Nitrophenyl- | 59 |
| | | 1,2,3-triazole-4-carboxylic acid 4- | isocyanate | |
| | | nitrophenylamide | | |
| | (b) | 1,2,3-Triazole-4-carboxylic acid 4- | | 58 |
| | | nitrophenylamide | | |
| 73 | (a) | 3-[2-(Trimethylsilyl)ethoxymethyl]- | 2-Nitro-4- | 34 |
| | | 1,2,3-triazole-4-carboxylic acid 2- | (trifluoromethyl)- | |
| | | nitro-4-(trifluoromethyl)phenylamide | phenylisocyanate | |
| | (b) | 1,2,3-Triazole-4-carboxylic acid 2- | | 74 |
| | | nitro-4-(trifluoromethyl)phenylamide | | |
| 74 | (a) | 3-[2-(Trimethylsilyl)ethoxymethyl]- | 4-Fluoro-2- | 48 |
| | | 1,2,3-triazole-4-carboxylic acid 4- | (trifluoromethyl)- | |
| | | fluoro-2- | phenylisocyanate | |
| | | (trifluoromethyl)phenylamide | | |
| | (b) | 1,2,3-Triazole-4-carboxylic acid 4- | | 39 |
| | | fluoro-2- | | |
| | | (trifluoromethyl)phenylamide | | |
| 75 | (a) | 3-[2-(Trimethylsilyl)ethoxymethyl]- | 4-Chloro-2- | 38 |
| | | 1,2,3-triazole-4-carboxylic acid 4- | (trifluoromethyl)- | |
| | | chloro-2- | phenylisocyanate | |
| | | (trifluoromethyl)phenylamide | | |
| | (b) | 1,2,3-Triazole-4-carboxylic acid 4- | | 76 |
| | | chloro-2- | | |
| | | (trifluoromethyl)phenylamide | | |
| 76 | (a) | 3-[2-(Trimethylsilyl)ethoxymethyl]- | 4-Chloro-2- | 38 |
| | | 1,2,3-triazole-4-carboxylic acid 4- | fluorophenylisocyanate | |
| | | chloro-2-fluorophenylamide | | |
| | (b) | 1,2,3-Triazole-4-carboxylic acid 4- | | 55 |
| | | chloro-2-fluorophenylamide | | |
| 77 | (a) | 3-[2-(Trimethylsilyl)ethoxymethyl]- | 2-Chloro-4- | 42 |
| | | 1,2,3-triazole-4-carboxylic acid 2- | (trifluoromethyl)- | |
| | | chloro-4- | phenylisocyanate | |
| | | (trifluoromethyl)phenylamide | | |
| | (b) | 1,2,3-Triazole-4-carboxylic acid 2- | | 73 |
| | | chloro-4- | | |
| | | (trifluoromethyl)phenylamide | | |
| 78 | (a) | 3-[2-(Trimethylsilyl)ethoxymethyl]- | 2-Fluoro-6- | 23 |
| | | 1,2,3-triazole-4-carboxylic acid 2- | (trifluoromethyl)phenylisocyanate | |
| | | fluoro-6- | | |
| | | (trifluoromethyl)phenylamide | | |
| | (b) | 1,2,3-Triazole-4-carboxylic acid 2- | | 60 |
| | | fluoro-6- | | |
| | | (trifluoromethyl)phenylamide | | |

TABLE 4
Physical properties of the compounds of Intermediates (a) 70 to 78 and
Examples (b) 70 to 78

| | | | MS | |
|---|---|---|---|---|
| | | | (M⁺ + 1), | |
| Ex. | Step | M.W. | m/z | ¹H NMR (DMSO-d₆, 400 MHz), δ |
| 70 | (a) | 421.78 | 421 | 10.83 (s, 1H), 8.48 (s, 1H), 7.86 (s, 2H), |
| | | | | 6.01 (s, 2H), 3.59 (t, 2H), 0.08 (t, 2H), −0.09 (s, 9H) |
| | (b) | 291.52 | 291 | 15.60 (br. s, 1H), 10.39 (s, 1H), 8.5 (s, 1H), |
| | | | | 7.80 (s, 2H) |
| 71 | (a) | 386.44 | 387 | 10.58 (s, 1H), 8.41 (s, 1H), 7.88-7.74 (m, 2H), |
| | | | | 7.64-7.49 (m, 2H), 6.00 (s, 2H), 3.58 (t, 2H), |
| | | | | 0.82 (t, 2H), −0.08 (s, 9H) |
| | (b) | 256.19 | 257 | 15.77 (br. s, 1H), 9.97 (s, 1H), 8.57 (s, 1H), |
| | | | | 7.84-7.71 (m, 3H), 7.49 (t, 1H) |
| 72 | (a) | 363.44 | 364 | 12.00 (s, 1H), 8.49 (s, 1H), 8.30 (d, 1H), |
| | | | | 7.99 (d, 1H), 6.02 (s, 2H), 3.59 (t, 2H), 0.82 (t, 2H), |
| | | | | −0.10 (s, 9H) |
| | (b) | 233.19 | 234 | 15.80 (br. s, 1H), 11.02 (s, 1H), 8.62 (s, 1H), |
| | | | | 8.26 (d, 2H), 8.13 (d, 2H) |
| 73 | (a) | 431.44 | 431 | 11.36 (s, 1H), 8.50 (s, 1H), 8.38 (d, 1H), |
| | | | | 8.19 (dd, 1H), 7.90 (d, 1H), 5.97 (s, 2H), 3.57 (t, |
| | | | | 2H), 0.82 (t, 2H), −0.09 (s, 9H) |
| | (b) | 301.19 | 302 | 15.94 (br. s, 1H), 11.66 (s, 1H), 8.71 (s, 1H), |
| | | | | 8.63 (d, 1H), 8.45 (d, 1H), 8.19 (dd, 1H) |
| 74 | (a) | 404.43 | 405 | 10.55 (br. s, 1H), 8.34 (s, 0.67H), 8.25 (s, |
| | | | | 033H), 7.77-7.52 (m, 3H), 5.96 (s, 2H), 5.68 (s, |
| | | | | 1H), 3.55 (t, 2H), 0.80 (t, 2H), −0.07 (s, 3H), |
| | | | | −0.09 (s, 6H) |
| | (b) | 274.18 | 275 | 15.74 (br. s, 1H), 10.04 (s, 1H), 8.54 (s, 1H), |
| | | | | 7.80-7.60 (m, 3H) |
| 75 | (a) | 420.89 | 421 | 10.63 (br. s, 1H), 8.40 (s, 1H), 7.11 (d, 1H), |
| | | | | 7.86 (dd, 1H), 7.59 (d, 1H), 5.99 (s, 2H), |
| | | | | 3.57 (t, 2H), 0.81 (t, 2H), −0.08 (s, 9H) |
| | (b) | 290.63 | 291 | 15.79 (br. s, 1H), 10.03 (s, 1H), 8.58 (s, 1H), |
| | | | | 7.87 (d, 1H), 7.83 (m, 2H) |
| 76 | (a) | 370.88 | 371 | 10.62 (s, 1H), 8.45 (s, 1H), 7.70-7.54 (m, 2H), |
| | | | | 7.36 (ddd, 1H), 6.00 (s, 2H), 3.58 (t, 2H), |
| | | | | 0.81 (t, 2H), −0.09 (s, 9H) |
| | (b) | 240.63 | 241 | 15.74 (br. s, 1H), 10.12 (s, 1H), 8.55 (s 1H), |
| | | | | 7.75 (t, 1H), 7.55 (dd, 1H), 7.32 (ddd, 1H) |
| 77 | (a) | 420.89 | 421 | 10.72 (br. s, 1H), 8.50 (s, 1H), 8.32 (s, 1H), |
| | | | | 7.88-7.78 (m, 2H), 6.01 (s, 2H), 3.59 (t, 2H), |
| | | | | 0.83 (t, 2H), −0.08 (s, 9H) |
| | (b) | 290.63 | 291 | 15.88 (br. s, 1H), 10.05 (s, 1H), 8.68 (s, 1H), |
| | | | | 8.32 (d, 1H), 8.02 (d, 1H), 7.80 (dd, 1H) |
| 78 | (a) | 404.43 | 405 | 10.64 (s, 1H), 8.46 (s, 1H), 7.80-7.65 (m, 3H), |
| | | | | 6.00 (s, 2H), 3.56 (t, 2H), 0.81 (t, 2H), −0.09 (s, |
| | | | | 9H) |
| | (b) | 274.18 | 275 | 15.73 (br. s, 1H), 10.20 (s, 1H), 8.52 (s, 1H), |
| | | | | 7.73-7.60 (m, 3H) |

### EXAMPLES 79-105

### General Procedure

Butyllithium (1.6 M in hexanes, 1.1 mL, 1.5 mmol) was added dropwise to a solution of 1-[2-(trimethylsilyl)ethoxymethyl]-1,2,3-triazole (3:1 mixture of the isomers, prepared as described hereinbefore, 210 μL, 299 mg, 1.5 mmol) in THF (12 mL) cooled to −50° C. The mixture was stirred at −50° C. for 30 min, cooled to −78° C. and a solution of the relevant isocyanate (2 mmol) in THF (5 mL) was added dropwise. The mixture was stirred at −78° C. for 30 min, allowed to warm to rt and stirred at rt for 16 h. The mixture was cooled to 0° C. and HCl (10 mL of 0.27M in EtOH, 2.7 mmol) was added. After stirring at 0° C. for 4 h, the mixture was concentrated and the residue purified by chromatography (eluent EtOAc/heptane, 20-60%) to give the title product.

TABLE 5
Examples (Ex.) 79 to 105

| | | | Yield |
|---|---|---|---|
| Ex. | Name | Arylisocyanate | % |
| 79 | 1,2,3-Triazole-4-carboxylic acid (4- | 4-Fluoro-3-methyl- | 27 |
| | fluoro-3-methylphenyl)amide | phenyl-isocyanate | |
| 80 | 1,2,3-Triazole-4-carboxylic | 2,3,4-Trifluoro- | 19 |
| | acid (2,3,4-trifluorophenyl)amide | methylphenyl- | |
| | | isocyanate | |
| 81 | 1,2,3-Triazole-4-carboxylic acid (2- | 2-Chloro-5-methyl- | 21 |
| | chloro-5-methylphenyl)amide | phenyliso-cyanate | |
| 82 | 1,2,3-Triazole-4-carboxylic acid (3,5- | 3,5-Dichlorophenyl- | 23 |
| | dichlorophenyl)amide | isocyanate | |
| 83 | 1,2,3-Triazole-4-carboxylic acid (2- | 2-Fluoro-5-methyl- | 14 |
| | fluoro-5-methylphenyl)amide | phenyliso-cyanate | |
| 84 | 1,2,3-Triazole-4-carboxylic acid (2- | 2-Chloro-6- | 28 |
| | chloro-6-trifluoromethylphenyl)amide | trifluoro- | |
| | | methylphenyliso- | |
| | | cyanate | |
| 85 | 1,2,3-Triazole-4-carboxylic acid (5- | 5-Chloro-2-methyl- | 28 |
| | chloro-2-methylphenyl)amide | phenyliso-cyanate | |
| 86 | 1,2,3-Triazole-4-carboxylic acid (3,5- | 3,5-Difluorophenyl- | 22 |
| | difluorophenyl)amide | isocyanate | |
| 87 | 1,2,3-Triazole-4-carboxylic acid (3,4- | 3,4-Difluorophenyl- | 29 |
| | difluorophenyl)amide | isocyanate | |
| 88 | 1,2,3-Triazole-4-carboxylic acid | 2-Fluoro-3- | 19 |
| | (2-fluoro-3- | trifluoromethyl- | |
| | trifluoromethylphenyl)amide | phenylisocyanate | |
| 89 | 1,2,3-Triazole-4-carboxylic acid (2,5- | 2,5-Difluorophenyl- | 34 |
| | difluorophenyl)amide | isocyanate | |
| 90 | 1,2,3-Triazole-4-carboxylic acid (2- | 2-Fluoro-5- | 29 |
| | fluoro-5-trifluoromethylphenyl)amide | trifluoromethyl- | |
| | | phenylisocyanate | |
| 91 | 1,2,3-Triazole-4-carboxylic acid (3- | 3-Fluoro-4-methyl- | 27 |
| | fluoro-4-methylphenyl)amide | phenylisocyanate | |
| 92 | 1,2,3-Triazole-4-carboxylic acid (3- | 3-Chloro-4-methyl- | 26 |
| | chloro-4-methylphenyl)amide | phenyliso-cyanate | |
| 93 | 1,2,3-Triazole-4-carboxylic acid (3- | 3-Fluoro-5- | 29 |
| | fluoro-5-trifluoromethylphenyl)amide | trifluoromethyl- | |
| | | phenylisocyanate | |
| 94 | 1,2,3-Triazole-4-carboxylic acid (4- | 4-Chloro-2-methyl- | 27 |
| | chloro-2-methylphenyl)amide | phenyliso-cyanate | |
| 95 | 1,2,3-Triazole-4-carboxylic acid (4- | 3-Trifluoromethyl- | 18 |
| | methyl-3- | 4-methylphenyliso- | |
| | trifluoromethylphenyl)amide | cyanate | |
| 96 | 1,2,3-Triazole-4-carboxylic acid (4- | 4-Trifluoromethoxy- | 10 |
| | trifluoromethoxyphenyl)amide | phenylisocyanate | |
| 97 | 1,2,3-Triazole-4-carboxylic acid (5- | 5-Flouro-2-methyl- | 23 |
| | fluoro-2-methylphenyl)amide | phenyliso-cyanate | |
| 98 | 1,2,3-Triazole-4-carboxylic acid | 3,4- | 20 |
| | (benzo[d][1,3]dioxol-5-yl)amide | Methylenedioxy- | |
| | | phenylisocyanate | |
| 99 | 1,2,3-Triazole-4-carboxylic acid (4- | 4-Chloro-3- | 15 |
| | chloro-3-trifluoromethylphenyl)amide | trifluoro- | |
| | | methylphenyl- | |
| | | isocyanate | |
| 100 | 1,2,3-Triazole-4-carboxylic acid (3- | 3-Chloro-4-fluoro- | 32 |
| | chloro-4-fluorophenyl)amide | phenylisocyanate | |
| 101 | 1,2,3-Triazole-4-carboxylic acid (3- | 3-Trifluoromethyl- | 22 |
| | trifluoromethylphenyl)amide | phenylisocyanate | |
| 102 | 1,2,3-Triazole-4-carboxylic acid (3- | 3-Chloro-2-methyl- | 21 |
| | chloro-2-methylphenyl)amide | phenyliso-cyanate | |
| 103 | 1,2,3-Triazole-4-carboxylic acid (4- | 4-Fluoro-3-trifluoro- | 4 |
| | fluoro-3-trifluoromethylphenyl)amide | methylphenyl- | |
| | | isocyanate | |
| 104 | 1,2,3-Triazole-4-carboxylic acid (2,6- | 2,6-Diisopropyl- | 25 |
| | diisopropylphenyl)amide | phenyl-isocyanate | |
| 105 | 1,2,3-Triazole-4-carboxylic acid [3,5- | 3,5-Bis(trifluoro- | 25 |
| | bis(trifluoromethyl)phenyl]amide | methyl)- | |
| | | phenylisocyanate | |

TABLE 6
Physical properties of the Examples 79-105

| | | MS | |
|---|---|---|---|
| | | (M⁺ + 1), | |
| Ex. | M.W. | m/z | ¹H NMR (DMSO-d₆, 400 MHz), δ |
| 79 | 220.20 | 221 | 10.41 (s, 1H), 8.55 (s, 1H), 7.78 (d, 1H), |
| | | | 7.71-7.65 (m, 1H), 7.16 (dd, 1H) |
| 80 | 242.16 | 243 | 10.44 (s, 1H), 8.62 (s, 1H), 7.53-7.36 (m, 2H) |
| 81 | 236.66 | 237 | 9.82 (s, 1H), 8.60 (br. s, 1H), 7.82 (br. s, 1H), 7.43 (d, |
| | | | 1H), 7.06 (dd, 1H), 2.32 (s, 1H) |
| 82 | 257.08 | 257 | 10.79 (s, 1H), 8.59 (br. s, 1H), 8.00-7.95 (m, 2H), |
| | | | 7.32 (dd, 1H) |
| 83 | 220.20 | 221 | 15.74 (br. s, 1H), 9.96 (s, 1H), 8.56 (br. s, 1H), |
| | | | 7.55 (d, 1H), 7.18 (dd, 1H), 7.03-7.07 (m, 1H), 2.30 (s, 3H) |
| 84 | 290.63 | 291 | 10.09 (s, 1H), 8.68 (br. s, 1H), 8.38 (d, 1H), 7.84 (d, |
| | | | 1H), 7.62 (dd, 1H) |
| 85 | 236.66 | 237 | 9.93 (s, 1H), 8.55 (br. s, 1H), 7.63 (dd, 1H), 7.32 (d, |
| | | | 1H), 7.21 (dd, 1H), 2.25 (s, 3H) |
| 86 | 224.17 | 225 | 10.82 (s, 1H), 8.58 (s, 1H), 7.63 (d, 2H), 6.95 (dd, 1H) |
| 87 | 274.17 | 225 | 10.75 (s, 1H), 8.63 (s, 1H), 8.05 (ddd, 1H), |
| | | | 7.76-7.70 (m, 1H), 7.49 (dd, 1H) |
| 88 | 274.17 | 275 | 10.38 (s, 1H), 8.59 (s, 1H), 8.00 (dd, 1H), 7.64 (dd, |
| | | | 1H), 7.44 (dd, 1H) |
| 89 | 224.17 | 225 | 10.14 (s, 1H), 8.66 (br. s, 1H), 7.73-7.78 (m, 1H), |
| | | | 7.39-7.47 (m, 1H), 7.13-7.20 (m, 1H) |
| 90 | 274.17 | 275 | 10.33 (s, 1H), 8.65 (s, 1H), 8.23 (d, 1H), |
| | | | 7.75-7.69 (m, 1H), 7.63 (dd, 1H) |
| 91 | 220.20 | 221 | 10.42 (s, 1H), 8.44 (s, 1H), 7.63 (d, 1H), 7.44 (d, 1H), |
| | | | 7.14 (dd, 1H) |
| 92 | 236.66 | 237 | 10.58 (s, 1H), 8.60 (s, 1H), 8.06 (d, 1H), 7.74 (dd, |
| | | | 1H), 7.38 (d, 1H) |
| 93 | 274.17 | 275 | 15.47 (br. s, 1H), 10.97 (s, 1H), 8.60 (s, 1H), 8.17 (s, |
| | | | 1H), 8.05 (d, 1H), 7.36 (d, 1H) |
| 94 | 236.66 | 237 | 9.94 (s, 1H), 8.52 (s, 1H), 7.49 (d, 1H), 7.37 (d, 1H), |
| | | | 7.27 (dd, 1H), 2.25 (s, 3H) |
| 95 | 270.21 | 271 | 10.68 (s, 1H), 8.55 (s, 1H), 8.25 (d, 1H), 7.99 (d, 1H), |
| | | | 7.41 (d, 1H) |
| 96 | 272.18 | 273 | 15.71 (br. s, 1H), 10.63 (s, 1H), 8.55 (s, 1H), 7.93 (d, |
| | | | 2H), 7.37 (d, 2H) |
| 97 | 220.20 | 221 | 9.85 (s, 1H), 8.55 (br. s, 1H), 7.48 (ddd, 1H), |
| | | | 7.30 (dd, 1H), 6.99 (ddd, 1H), 2.25 (s, 3H) |
| 98 | 232.20 | 233 | 10.30 (s, 1H), 8.48 (br. s, 1H), 7.46 (d, 1H), 7.27 (dd, |
| | | | 1H), 6.88 (d, 1H), 6.00 (s, 1H) |
| 99 | 190.63 | 291 | 15.82 (br. s, 1H), 10.92 (s, 1H), 8.59 (s, 1H), 8.46 (d, |
| | | | 1H), 8.16 (dd, 1H), 7.73 (d, 1H) |
| 100 | 240.62 | 241 | 10.68 (s, 1H), 8.56 (s, 1H), 8.12 (dd, 1H), 7.81 (ddd, |
| | | | 1H), 7.41 (dd, 1H) |
| 101 | 256.18 | 257 | 10.77 (s, 1H), 8.57 (s, 1H), 8.31 (s, 1H), 8.11 (d, 1H), |
| | | | 7.59 (dd, 1H), 7.45 (d, 1H) |
| 102 | 236.66 | 237 | 10.19 (s, 1H), 8.53 (br. s, 1H), 7.42-7.34 (m, 2H), |
| | | | 7.25 (dd, 1H) |
| 103 | 274.17 | 275 | 15.87 (br. s, 1H), 10.90 (s, 1H), 8.65 (s, 1H), 8.42 (dd, |
| | | | 1H), 8.28-8.20 (m, 1H), 7.59 (dd, 1H) |
| 104 | 272.35 | 273 | 9.89 (s, 1H), 8.47 (s, 1H), 7.27 (dd, 1H), 7.20 (d, 2H), |
| | | | 3.08 (heptet, 2H), 1.13 (d, 12H) |
| 105 | 324.18 | 325 | 11.15 (s, 1H), 8.48 (s, 1H), 8.40 (s, 2H), 7.90 (s, 1H), |
| | | | 6.02 (s, 2H), 3.59 (dd, 2H), 0.82 (s, 2H), −0.11 (s, 9H) |

### EXAMPLE 106

Title-compounds of the examples were tested in the biological test described above and were found to exhibit an IC₅₀ of below 10 pA For example, the following representative compounds of the examples exhibited the following IC₅₀ values:

### Example 1: 1400 nM

### Example 6: 330 nM

### Example 7: 1800 nM

### Example 9: 1600 nM

### Example 12: 760 nM

### Example 18: 950 nM

### Example 35: 810 nM

### Example 36: 1160 nM

### Example 73: 3800 nM

### Example 75: 250 nM

### Example 76: 530 nM

### Example 82: 4100 nM

Example 91: 9400 nM

1. A compound of formula I, 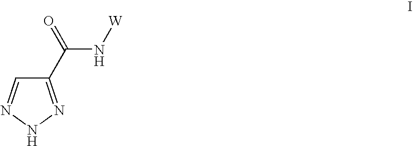
wherein
W represents an aryl or heteroaryl group, optionally substituted by one or more substituents selected from:
1) G¹;
2) aryl or heteroaryl, both of which are optionally substituted by one or more substituents selected from A¹, —N₃, —NO₂ and —S(O)ₚR^{6e}; and
3) heterocycloalkyl, which is optionally substituted by one or more substituents selected from A², —N₃, —NO₂ and ═O;
G¹ represents halo, —R^{3a}, —CN, —C(O)R^{3b}, —C(O)OR^{3c}, —C(O)N(R^{4a})R^{5a}, —N(R^{4b})R^{5b}, N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h}, —S(O)₂OH, —P(O)(OR⁴ⁱ)(OR⁵ⁱ) or —C(O)N(R^{3q})S(O)₂R^{3r};
R^{3a} represents C₁₋₆ alkyl optionally substituted by one or more substituents selected from Z, F, Cl, —N(R^{6b})R^{6c}, —N₃, ═O and —OR^{6d};
R^{3b}, R^{3c}, R^{3h}, R³ⁿ and R^{4a} to R^{4h} independently represent H, Z or C₁₋₆ alkyl optionally substituted by one or more halo atoms or —OR^{6d};
R^{3d} to R^{3g}, R^{3k}, R^{3q}, R^{5a}, R^{5b}, R^{5d} and R^{5f} to R^{5h} independently represent H or C₁₋₆ alkyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h} may be linked together to form a 3- to 6-membered ring, which ring optionally contains a further heteroatom in addition to the nitrogen atom to which these substituents are necessarily attached, and which ring is optionally substituted by ═O or C₁₋₆ alkyl optionally substituted by one or more fluoro atoms;
R³ⁱ, R^{3j}, R^{3m}, R^{3p} and R^{3r} independently represent Z or C₁₋₆ alkyl optionally substituted by one or more substituents selected from B¹;
R⁴ⁱ and R⁵ⁱ independently represent H or C₁₋₆ alkyl optionally substituted by one or more substituents selected from B²;
Z represents:
a) heterocycloalkyl optionally substituted by one or more substituents selected from A³ and ═O;
b) aryl or heteroaryl both of which are optionally substituted by one or more substituents selected from A⁴, —N₃, —NO₂ and —S(O)_{q}R^{7e};
A¹, A², A³ and A⁴ independently represent halo, R^{6a}, —CN, —N(R^{6b})R^{6c} or —OR^{6d};
R^{6b} to R^{6d} independently represent H or C₁₋₆ alkyl optionally substituted by one or more substituents selected from B³;
R^{6a}, R^{6e} and R^{7e} independently represent C₁₋₆ alkyl optionally substituted by one or more substituents selected from B⁴; or
R^{6b} and R^{6c} may be linked together to form a 3- to 6-membered ring, which ring optionally contains a further heteroatom in addition to the nitrogen atom to which these substituents are necessarily attached, and which ring is optionally substituted by ═O or C₁₋₆ alkyl optionally substituted by one or more fluoro atoms;
B¹, B², B³ and B⁴ independently represent F, Cl, —OCH₃, —OCH₂CH₃, —OCHF₂, —OCH₂CF₃, —OCF₃ or —OCF₂CF₃; and
m, p and q independently represent 0, 1 or 2,
or a pharmaceutically-acceptable salt thereof,
provided that:
(A) when W represents a phenyl group substituted by one G¹ substituent at the ortho position,
G¹ represents R^{3a}, R^{3a} represents ethynyl substituted by Z, Z represents 2-thiazolyl substituted in the 4-position by A⁴, A⁴ represents R^{6a}, then R^{6a} does not represent cyclobutyl;
(B) when W represents a 6-quinazolinyl group substituted in the 4-position by G¹, G¹ represents —N(R^{4b})R^{5b}, R^{5b} represents H and R^{4b} represents Z, then Z does not represent 3-chloro-4-fluorophenyl.

2. A compound as claimed in claim 1, wherein W represents an optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, oxindolyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, benzothiazolyl, 1,4-benzodioxanyl, 1,3,4-oxadiazolyl or 1,3,4-thiadiazolyl, group.

3. A compound as claimed in claim 2, wherein W represents optionally substituted thiazolyl, 1,3-benzodioxolyl, pyrimidinyl, quinoxalinyl, quinolinyl, phenyl or pyridyl.

4. A compound as claimed in claim 3, wherein W represents optionally substituted quinoxalinyl, quinolinyl, phenyl or pyridyl.

5. A compound as claimed in claim 1, wherein W is optionally substituted by between 1 and 4 substituents selected from aryl and G¹.

6. A compound as claimed in claim 1, wherein, when W is substituted, then it is substituted by one to three substituents selected from G¹.

7. A compound as claimed in claim 1, wherein G¹ represents halo, —R^{3a}, —CN, —C(O)R^{3b}, —C(O)OR^{3c}, —C(O)N(R^{4a})R^{5a}, —N(R^{4b})R^{5b}, N(R^{3d})C(O)R^{4c}, —N(R^{3e})C(O)N(R^{4d})R^{5d}, —N(R³¹)C(O)OR^{4e}, —NO₂, N(R^{3g})S(O)₂N(R^{4f})R^{5f}, OR^{3h}, OC(O)N(R^{4g})R^{5g}, —OS(O)₂R³ⁱ, S(O)ₘR^{3j} or —S(O)₂N(R^{4h})R^{5h}.

8. A compound as claimed in claim 1, wherein, when any of the pairs R^{4a} and R^{5a}, R^{4b} and R^{5b}, R^{4d} and R^{5d}, R^{4f} and R^{5f}, R^{4g} and R^{5g}, or R^{4h} and R^{5h}, are linked together, they form a 5- to 6-membered ring, which ring optionally contains a further heteroatom and is optionally substituted by methyl, —CHF₂, —CF₃ or ═O.

9. A compound as claimed in claim 1, wherein R^{3a} represents C₁₋₆ alkyl optionally substituted by one or more substituents selected from F and —OR^{6d}.

10. A compound as claimed in claim 9, wherein R^{3a} represents C₁₋₃ alkyl optionally substituted by one or more fluoro atoms.

11. A compound as claimed in claim 1, wherein R^{3b}, R^{3c}, R^{3h}, R^{4a} to R^{4h}, R^{5a}, R^{5b}, R^{5d}, R^{5f} to R^{5h} independently represent H or optionally substituted C₁₋₄ alkyl or the relevant pairs are linked together.

12. A compound as claimed in claim 11, wherein R^{3h} represents hydrogen or C₁₋₄ alkyl optionally substituted by one or more fluoro atoms.

13. A compound as claimed in claim 11 or claim 12, wherein R^{4b} and R^{5b} independently represent C₁₋₂ alkyl.

14. A compound as claimed in claim 1, wherein R^{3d} to R^{3g} independently represent C₁₋₄ alkyl or H.

15. A compound as claimed in claim 1, wherein R³ⁱ and R^{3j} independently represent C₁₋₄ alkyl optionally substituted by one or more B¹ substituents.

16. A compound as claimed in claim 1, wherein B¹ represents F.

17. A compound as claimed in claim 1, wherein the optional substituents on W are aryl, —N(R^{3f})C(O)OR^{4e}, —S(O)₂N(R^{4h})R^{5h}, halo, —R^{3a}, —OR^{3h} or —NO₂.

18. A compound as claimed in claim 17, wherein the optional substituents are halo, —R^{3a}, —OR^{3h} or —NO₂.

19. A compound as claimed in claim 17, wherein the optional substituents on W are phenyl, bromo, ethyl, propyl, —NHC(O)Ot-butyl, ethoxy, propoxy, butoxy, trifluoromethoxy, —S(O)₂NH₂, —S(O)₂N(CH₃)H, —S(O)₂N(CH₃)₂, —S(O)₂N(CH₂CH₃)₂, isopropyl, fluoro, chloro, methyl, methoxy, —NO₂ or trifluoromethyl.

20. A compound as claimed in claim 18 or claim 19, wherein the optional substituents on W are fluoro, chloro, methyl, methoxy, —NO₂ or trifluoromethyl.

21. A compound of formula I as defined in claim 1, or a pharmaceutically acceptable salt thereof, for use as a pharmaceutical.

22. A pharmaceutical formulation including a compound of formula I, as defined in claim 1, or a pharmaceutically acceptable salt thereof, in admixture with a pharmaceutically acceptable adjuvant, diluent or carrier.

23. (canceled)

24. A method as claimed in claim 27 wherein the lipoxygenase is 15-lipoxygenase.

25. A method as claimed in claim 24, wherein the disease is inflammation and/or has an inflammatory component.

26. A method as claimed in claim 25 wherein the inflammatory disease is asthma, chronic obstructive pulmonary disease, pulmonary fibrosis, an allergic disorder, rhinitis, inflammatory bowel disease, an ulcer, inflammatory pain, fever, atherosclerosis, coronary artery disease, vasculitis, pancreatitis, arthritis, osteoarthritis, rheumatoid arthritis, conjunctivitis, iritis, scleritis, uveitis, a wound, dermatitis, eczema, psoriasis, stroke, diabetes, autoimmune diseases, Alzheimer's disease, multiple sclerosis, sarcoidosis, Hodgkin's disease or another malignancy.

27. A method of treatment of a disease in which inhibition of the activity of a lipoxygenase is desired and/or required, which method comprises administration of a therapeutically effective amount of a compound of formula I as defined in claim 1 but without the provisos, or a pharmaceutically-acceptable salt thereof, to a patient suffering from, or susceptible to, such a condition.

28. A combination product comprising:
(A) a compound of formula I as defined in claim 1 but without the provisos, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

29. A combination product as claimed in claim 28 which comprises a pharmaceutical formulation including a compound of formula I but without the provisos, or a pharmaceutically-acceptable salt thereof, another therapeutic agent that is useful in the treatment of inflammation, and a pharmaceutically-acceptable adjuvant, diluent or carrier.

30. A combination product as claimed in claim 28 which comprises a kit of parts comprising components:
(a) a pharmaceutical formulation including a compound of formula I but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

31. A process for the preparation of a compound of formula I as defined in claim 1, which comprises:
(i) reaction of 1,2,3-triazole-4-carboxylic acid, or a N-protected and/or O-protected derivative thereof, with a compound of formula II,
WNH₂ II
wherein W is as defined in claim 1;
(ii) reaction of 1,2,3-triazole-4-carboxylic acid amide, or a N-protected derivative thereof, with a compound of formula III,
W-L¹ III
wherein L¹ represents a suitable leaving group and W is as defined in claim 1;
(iii) reaction of a compound of formula IV, 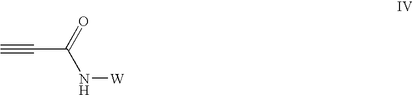
wherein W is as defined in claim 1, or a N-protected derivative thereof, with a suitable reagent that provides a source of azide ions;
(iv) reaction of triazole, or a protected derivative thereof, with an appropriate base, followed by reaction with a compound of formula V,
W—N═C═O V
wherein W is as defined in claim 1, followed by quenching with a suitable proton source; or
(v) reaction of a compound of formula VI, 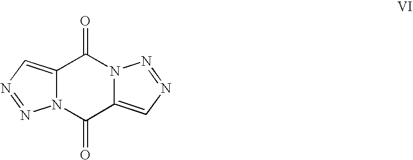
with a compound of formula II as defined above.

32. A process for the preparation of a pharmaceutical formulation as defined in claim 22, which process comprises bringing into association a compound of formula I, or a pharmaceutically acceptable salt thereof with a pharmaceutically-acceptable adjuvant, diluent or carrier.

33. A process for the preparation of a combination product as defined in claim 28, which process comprises bringing into association a compound of formula I, but without the provisos, or a pharmaceutically acceptable salt thereof with the other therapeutic agent that is useful in the treatment of inflammation, and at least one pharmaceutically-acceptable adjuvant, diluent or carrier.
